(19) United States
(12) Patent Application Publication
(10) Pub. No.: US 2010/0160391 A1
(43) Pub. Date: Jun. 24, 2010

(54) Substituted Aminopyridines as Fluorescent Reporters for Amide Hydrolases

(76) Inventors: Bruce D. Hammock, Davis, CA (US); Huazhang Huang, Davis, CA (US); Kosuke Nishi, Gwangju (KR)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/373,239

(22) PCT Filed: Jul. 9, 2007
(86) PCT No.: PCT/US07/73076

§ 371 Date: Oct. 9, 2009

Related U.S. Application Data
(60) Provisional application No. 60/819,973, filed on Jul. 10, 2006.

(51) Int. Cl. A61K 31/44; C07D 213/72; C07K 2/00; C12Q 1/34

(52) U.S. Cl. 514/352; 546/304; 530/300; 530/409; 435/18

(57) ABSTRACT

The present invention provides conjugates comprising a substituted aminopyridine covalently attached to an organic molecule via an amide bond. Such conjugates find utility as substrates for amide hydrolases, where the substituted aminopyridine acts as a fluorescent reporter of amide hydrolase activity. As a result, the conjugates described herein can advantageously be used in assays to detect amide hydrolase activity based upon measuring the fluorescence of a substituted aminopyridine that is released after amide hydrolysis. The conjugates of the present invention are also particularly useful in screening assays, which enable the identification of inhibitory molecules for amide hydrolases and other enzymes. The identified amide hydrolase inhibitors can be used in the treatment of a variety of diseases and disorders associated with aberrant amide hydrolase activity.

### BACKGROUND OF THE INVENTION

Amide hydrolases are enzymes that catalyze the hydrolysis of acid amides in a variety of substrates ranging from lipids to polypeptides. For example, fatty acid amide hydrolase (FAAH) is a mammalian integral membrane enzyme that plays a critical role in regulating the levels of endogenous signaling lipids such as the cannabinoid anandamide, the sleeping-inducing substance oleamide, the anorexigenic compound N-oleoylethanolamide, and the anti-inflammatory agent N-palmitoylethanolamide (McKinney et al., Annu. Rev. Biochem., 74:411-432 (2005); Maurelli et al., FEBS Lett., 377:82-87 (1995); Boger et al., Bioorg. Med. Chem. Lett., 10:2613-2616 (2000); Rodríguez de Fonseca et al., Nature, 414:209-212 (2001); Goparaju et al., Biochem. Pharmacol., 57:417-423 (1999)). Inhibition of FAAH is associated with therapeutic benefits such as hypoalgesia, relief of pain and spasticity, relief of anxiety, and protection from inflammation (Cravatt et al., Proc. Natl. Acad. Sci. USA, 98:9371-9376 (2001); Massa et al., J. Clin. Invest., 113:1202-1209 (2004); Cravatt et al., Proc. Natl. Acad. Sci. USA, 101:10821-10826 (2004); Kathuria et al., Nat. Med., 9:76-81 (2003); Lichtman et al., Pain, 109:319-327 (2004); Walker et al., Proc. Natl. Acad. Sci. USA, 96:12198-12203 (1999); Cravatt et al., Curr. Opin. Chem. Biol., 7:469-475 (2003); Bisogno et al., Curr. Pharm. Des., 8:533-547 (2002); Cravatt et al., Proc. Natl. Acad. Sci. USA, 98:9371-9376 (2001); Hohmann et al., Nature, 435:1108-1112 (2005)). Therefore, the identification of compounds that inhibit FAAH is of medical and therapeutic significance.

To meet the increasing demands of identifying novel inhibitors from large libraries of chemical compounds, simple yet highly sensitive and specific assays which are suitable for a high-throughput format using colorimetric or fluorescent detection in 96- or 384-well plates are crucial for screening. Most assays for FAAH activity are commonly associated with the use of radiolabeled substrates or chromatographic techniques, which are costly, labor-intensive, and not compatible with the high-throughput format or contain some other limitation (Deutsch et al., Biochem. Pharmacol., 46:791-796 (1993); Maccarrone et al., Anal. Biochem., 267:314-318 (1999); Thumser et al., Biochem. Pharmacol., 53:433-435 (1997); Wilson et al., Anal. Biochem., 318:270-275 (2003); Qin et al., Anal. Biochem., 261:8-15 (1998); Patterson et al., J. Am. Chem. Soc., 118:5938-5945 (1996); Desarnaud et al., J. Biol. Chem., 270:6030-6035 (1995); Koutek et al., J. Biol. Chem., 269:22937-22940 (1994); Omeir et al., Life Sci., 56:1999-2005 (1995)). Recently, two assays that are suitable for high-throughput formats such as 96 or 384-well plate assays have been reported (De Bank et al., Anal. Biochem., 69:1187-1193 (2005); Ramarao et al., Anal. Biochem., 343:143-151 (2005)). One is a colorimetric, dual-enzyme assay that utilizes the ability of FAAH to hydrolyze oleamide and measures the production of ammonia by an NADH/NAD⁺-coupled enzyme (De Bank et al., supra). Its application, however, is limited by a lack of sensitivity and the usage of another enzyme (i.e., L-glutamate dehydrogenase). The other method is a fluorescent assay using arachidonyl 7-amino-4-methylcoumarin amide as a substrate (Ramarao et al., supra). However, the application of this assay is limited by a lack of sensitivity, poor aqueous solubility, and substrate instability.

2-, 3-, and 4-aminopyridines are generally known to be fluorescent compounds (Weisstuch et al., J. Phys. Chem., 72:1982-1987 (1968); Rusakowicz et al., J. Phys. Chem., 72:2680-2681 (1968)). Among these simple aminopyridines, the 2- and 3-isomers show a relatively stronger fluorescence than the 4-isomer. Therefore, both 2- and 3-isomers have been used as fluorescent reporters of hydrolytic activities of multiple enzymes such as nucleotide pyrophosphatase (Anderson et al., Mol. Cell. Biochem., 8:89-96 (1975), transferases (Kato et al., JP 06065300; Uozumi et al., J. Biochem., 120:385-392 (1996)), and hyaluronidase (Nakamura et al., Anal. Biochem., 191:21-24 (1990)). However, the fluorescent strength (or quantum yield) of these aminopyridines is relatively low compared to 7-aminocoumarin and greatly varies with the change of pH values in media due to protonization or deprotonization of both nitrogen atoms (Weisstuch et al., supra). Generally, strong fluorescence for simple aminopyridines is shown in acidic or neutral aqueous media, but weak fluorescence in basic aqueous media. These properties severely limit the application of simple aminopyridines in enzymatic assays involving amide hydrolases, which show maximal hydrolytic activities in basic aqueous media.

As such, there is a need in the art for the development of novel and highly sensitive fluorescent substrates for amide hydrolases. The present invention satisfies this and other needs.

### BRIEF SUMMARY OF THE INVENTION

The present invention provides conjugates comprising a substituted aminopyridine covalently attached to an organic molecule via an amide bond. Such conjugates find utility as substrates for amide hydrolases, where the substituted aminopyridine acts as a fluorescent reporter of amide hydrolase activity. As a result, the conjugates described herein can advantageously be used in assays to detect amide hydrolase activity based upon measuring the fluorescence of a substituted aminopyridine that is released after amide hydrolysis. The conjugates of the present invention are also particularly useful in screening assays, which enable the identification of inhibitory molecules for amide hydrolases and other enzymes.

The identified amide hydrolase inhibitors can be used in the treatment of a variety of diseases and disorders associated with aberrant amide hydrolase activity.

In one aspect, the present invention provides a conjugate comprising:
(a) a substituted aminopyridine having the formula:

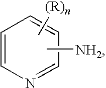

wherein R is n independently selected substituted groups and n is 1, 2, 3, or 4; and
(b) an organic molecule covalently attached to the —NH₂ group of the substituted aminopyridine via an amide bond.

In some embodiments, at least one of the substituted groups comprises an alkoxy group, a substituted amino group, or other electron donor group. Examples of alkoxy groups include, but are not limited to, a methoxy group, an ethoxy group, an aryloxy group, and a t-butoxy group. Non-limiting examples of substituted amino groups include dialkylamino groups such as a dimethylamino group. In other embodiments, each of the substituted groups present on the substituted aminopyridine is located para, ortho, or meta to the —NH₂ group.

In preferred embodiments, n is 1 and the substituted group comprises a methoxy group or a dimethylamino group that is para to the —NH₂ group.

One of skill in the art will appreciate that any organic molecule can be conjugated to a substituted aminopyridine through the formation of an amide bond. Suitable amide bonds include, without limitation, a carboxamide bond (—HN—CO), a sulfonamide bond (—HN—SO₂), and a phosphonamide bond (—HN—PO₂). Non-limiting examples of organic molecules include fatty acids, lipids, amino acids, peptides, polypeptides, proteins, glycoproteins, small organic molecules, polysaccharides, oligosaccharides, polynucleotides, and oligonucleotides.

In certain embodiments, the conjugate is a substrate for an amide hydrolase. The amide hydrolase is generally a short- or long-chain fatty acid amide hydrolase, a linear amide hydrolase, a cyclic amide hydrolase, or a peptide hydrolase. In some instances, the organic molecule present in the conjugate comprises a fatty acid and the amide hydrolase comprises a fatty acid amide hydrolase (FAAH). In other instances, the organic molecule present in the conjugate comprises a proteinaceous compound (e.g., an amino acid, a peptide, a peptoid, a peptidomimetic, a polypeptide, a protein, etc.) and the amide hydrolase comprises a peptide hydrolase such as an aminopeptidase (e.g., L-Leucine aminopeptidase).

In certain other embodiments, the conjugate is a substrate for a transferase. In some instances, the conjugate comprises a sulfonamide bond that is cleaved by glutathione-S-transferase (GST). Non-limiting examples of glutathione-S-transferases include any of the isoenzymes in the α, μ, π, σ, or θ class. In one embodiment, the conjugate comprises the following structure:

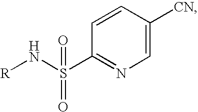

wherein R is a substituted aminopyridine as described herein. Additional examples of organic molecules which can be covalently attached to substituted aminopyridines and used as substrates for glutathione-S-transferase in accordance with the present invention are described in Koeplinger et al., Drug Metab. Dispos., 27:986-991 (1999) and Zhao et al., Drug Metab. Dispos., 27:992-998 (1999).

In another aspect, the present invention provides a method for determining amide hydrolase activity, the method comprising:
(a) contacting an amide hydrolase with a conjugate described herein; and
(b) measuring a level of substituted aminopyridine released from the conjugate by the amide hydrolase.

In some embodiments, the level of released substituted aminopyridine is measured using fluorescence detection. For example, the amount of fluorescence in a sample can be measured using a fluorometer such as a filter fluorometer or a spectrofluorometer, wherein excitation radiation from an excitation source having a first wavelength passes through excitation optics and causes the excitation radiation to excite the sample. In response, free (i.e., released) substituted aminopyridine molecules present in the sample emit radiation having a wavelength that is different from the excitation wavelength. Collection optics then collect the emission from the sample. The device can include a temperature controller to maintain the sample at a specific temperature while it is being scanned, and can have a multi-axis translation stage, which moves a microtiter plate holding a plurality of samples in order to position different wells to be exposed. The multi-axis translation stage, temperature controller, auto-focusing feature, and electronics associated with imaging and data collection can be managed by an appropriately programmed digital computer, which can also transform the data collected during the assay into another format for presentation. This process can be miniaturized and automated to enable screening many thousands of compounds in a high-throughput format. These and other methods of performing assays on fluorescent materials are well known in the art (see, e.g., Lakowicz, “Principles of Fluorescence Spectroscopy,” Plenum Press (1983); Herman, “Resonance energy transfer microscopy,” In “Fluorescence Microscopy of Living Cells in Culture,” Part B, Meth. Cell Biol., 30:219-243, Ed. Taylor and Wang, Academic Press (1989); and Turro, “Modern Molecular Photochemistry,” Benjamin/Cummings Publ. Co., Inc., pp. 296-361 (1978)).

In other embodiments, the level of released substituted aminopyridine is associated with a disease or disorder. Examples of such diseases or disorders include, but are not limited to, a neurological disorder, an inflammatory disease, an autoimmune disease, a circulatory disease, a liver disease, and cancer. In one embodiment, an increased level of substituted aminopyridines released from the conjugate compared to a control sample can be indicative of aberrant (e.g., elevated or undesirable) amide hydrolase activity. In certain instances, the control sample can comprise a sample of amide hydrolase obtained from a healthy individual.

In yet another aspect, the present invention provides a method for identifying a compound that inhibits an amide hydrolase, the method comprising:
(a) contacting an amide hydrolase with a conjugate described herein and a compound; and
(b) determining the effect of the compound on amide hydrolase activity, thereby identifying a compound that inhibits the amide hydrolase.

In some embodiments, the effect of the compound on amide hydrolase activity is determined by measuring a level of substituted aminopyridine released from the conjugate by the amide hydrolase. The level of released substituted aminopyridine is typically measured using fluorescence detection, e.g., a fluorometer. In other embodiments, the screening method of the present invention further comprises comparing the level of released substituted aminopyridine in the presence of the compound relative to the absence of the compound. In certain instances, a decrease in the level of released substituted aminopyridine indicates that the compound inhibits the amide hydrolase.

In a related aspect, the present invention provides a method of inhibiting an amide hydrolase in a subject by administering to the subject a therapeutically effective amount of a compound identified by the screening method described herein.

Also provided is a kit comprising a conjugate described herein and directions for use of the conjugate in determining amide hydrolase activity. The present invention further provides a kit comprising a conjugate described herein and directions for use of the conjugate in identifying a compound that inhibits an amide hydrolase.

One of skill in the art will appreciate that the conjugates of the present invention can also be used in assays and kits for determining glutathione-S-transferase activity. As a non-limiting example, the level of substituted aminopyridine released from the conjugate by a glutathione-S-transferase can be associated with the ability of that glutathione-S-transferase to cleave a sulfonamide bond present in an organic molecule such as a xenobiotic (e.g., a drug, a prodrug, a poison, or other compound). Additionally, one of skill in the art will understand that the conjugates of the present invention can be used in screening assays and kits to identify glutathione-S-transferases which are capable of cleaving and activating prodrugs containing sulfonamide bonds (e.g., HIV protease inhibitors, anticancer compounds, and the like) to their active metabolites.

Other objects, features, and advantages of the present invention will be apparent to one of skill in the art from the following detailed description and figures.

### BRIEF DESCRIPTION OF THE DRAWINGS

FIG. 1 shows the structures of the fluorescent reporters and substrates used in Example 1.

FIG. 2 shows the fluorescent spectra of 5-amino-2-methoxypyridine (6) and its amide derivative, N-(6-methoxypyridin-3-yl) octanamide (13). The dashed curve on the left side is the excitation wavelength of (6) from 250 to 350 nm, which was determined using 25 μM of (6) in 0.1 M sodium phosphate buffer (pH 8.0) containing 1% ethanol at a fixed emission (396 nm) and room temperature. The five curves from top to bottom on the right side are curves of the emission wavelengths of (6) at pH 9 (Tris/HCl buffer), 8, 7, and 6, and (13) at pH 8, respectively. They were determined using a 25 μM final concentration of (6) or (13) in 0.1 M sodium phosphate buffer (pH 6-8) or Tris/HCl (pH 9.0) in a 1-cm cuvette at a fixed excitation wavelength (302 nm) and room temperature.

FIG. 3 shows the effect of the amount of bovine serum albumin (BSA) on the relative fluorescent intensity of 5-amino-2-methoxypyridine (6) at 37° C. Assays were conducted in 200 μl of 0.1 M sodium phosphate buffer (pH 8.0) containing different concentrations of (6) (final concentration=0, 0.4, 0.78, 1.56, 3.13, 6.25, 12.5, or 25 μM) and BSA (0 1, 10, or 100 μg/well). Relative fluorescent intensity was recorded at an excitation wavelength of 302 nm and an emission wavelength of 396 nm.

FIG. 4 shows a comparison of the aqueous solubility of the coumarin substrate (15) and the pyridine substrates (13) and (14). Assays were performed in 200 μl of 0.1 M sodium phosphate buffer (pH 8.0) with 1% ethanol and different concentrations of the substrate (final concentration=0.39, 0.78, 1.56, 3.13, 6.25, 12.5, 25, 50, 100, or 200 μM). Relative absorbance was recorded at a wavelength of 800 nm at 37° C.

FIG. 5 shows a comparison of the kinetic data of fatty acid amide hydrolase (FAAH) toward substrates (13) and (17). Assays were performed in 200 μl of 0.1 M sodium phosphate buffer (pH 8.0) with 1% DMSO and different concentrations of the substrate (final concentration=0.78, 1.56, 3.13, 6.25, 12.5, 25, 50, 100, or 200 μM of (13) and 0.39, 0.78, 1.56, 3.13, 6.25, 12.5, 25, 50, or 100 μM of (17) at 37° C. Data were analyzed by the SigmaPlot Enzyme Kinetics software (2001) using the Michaelis-Menten equation for all concentrations.

FIG. 6 shows the structures of the fluorescent and colorimetric substrates used in Example 2.

FIG. 7 shows the excitation and emission spectra of a red-shifted substituted aminopyridine of the present invention.

### DETAILED DESCRIPTION OF THE INVENTION

### I. Introduction

The present invention provides conjugates comprising a substituted aminopyridine (e.g., a substituted 3- or 5-aminopyridine) covalently attached to an organic molecule via an amide bond. Such conjugates are suitable as substrates for amide hydrolases (e.g., fatty acid amide hydrolase (FAAH), aminopeptidase, etc.), where the substituted aminopyridine acts as a fluorescent reporter of amide hydrolase activity. As a result, the conjugates described herein can advantageously be used in simple, novel, highly sensitive, and continuous fluorescent assays to detect amide hydrolase activity based upon measuring the strong fluorescence of a substituted aminopyridine that is liberated (i.e., released) after amide hydrolysis. In particular, the fluorescent assays described herein are sufficiently robust, efficient, and low-cost to allow the identification of inhibitory molecules for amide hydrolases and other enzymes that can be useful in a variety of therapeutic applications.

As a non-limiting example, the screening methods of the present invention find utility in identify inhibitors of fatty acid amide hydrolase (FAAH) activity. FAAH, an integral membrane protein that is widely expressed in brain and other tissues, hydrolyzes bioactive amides including the endocannabinoid anandamide as well as other simple esters and amides with long unsaturated acyl chains. It has been demonstrated in FAAH(−/−) mice that marked depression of FAAH activity results in reduced sensation of pain and enhanced endocannabinoid signalling. Thus, compounds identified using the substituted aminopyridine conjugates described herein which inhibit FAAH activity can find utility as analgesic and anxiolytic drugs in the treatment of various neurological disorders.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice the present invention. For purposes of the present invention, the following terms are defined.

The term “amide hydrolase” as used herein refers to an enzyme that catalyzes the cleavage of an amide bond in an organic molecule such as a peptide, polypeptide, protein, lipid, nucleic acid, oligosaccharide, and the like. Amide hydrolases typically belong to an enzyme class having an E.C.3.-.-.- (hydrolase) Enzyme Commission (E.C.) classification number (Nomenclature Committee of the International Union of Biochemistry and Molecular Biology). See, e.g., http://www.ebi.ac.uk/thornton-srv/databases/enzymes/ for a description of enzymes whose activity can be assayed using the compositions and methods of the present invention.

Examples of amide hydrolases include, but are not limited to, short- or long-chain fatty acid amide hydrolases such as fatty acid amide hydrolase (FAAH) and N-palmitoylethanolamine-selective acid amidase (NPAA).

Amide hydrolases also include, for example, linear amide hydrolases such as asparaginase, glutaminase, omega-amidase, amidase, urease, beta-ureidopropionase, ureidosuccinase, formylaspartate deformylase, arylformamidase, formyltetrahydrofolate deformylase, penicillin amidase, biotimidase, aryl-acylamidase, aminoacylase, aspartoacylase, acetylornithine deacetylase, acyl-lysine deacylase, succinyl-diaminopimelate desuccinylase, nicotinamidase, citrullinase, N-acetyl-beta-alanine deacetylase, pantothenase, ceramidase, choloylglycine hydrolase, N-acetylglucosamine-6-phosphate deacetylase, N(4)-(beta-N-acetylglucosaminyl)-L-asparaginase, N-formylmethionylaminoacyl-tRNA deformylase, N-acetylmuramoyl-L-alanine amidase, 2-(acetamidomethylene)succinate hydrolase, 5-aminopentanamidase, formylmethionine deformylase, hippurate hydrolase, N-acetylglucosamine deacetylase, D-glutaminase, N-methyl-2-oxoglutaramate hydrolase, glutamin-(asparagin-)ase, alkylamidase, acylagmatine amidase, chitin deacetylase, nicotinamide-nucleotide amidase, peptidyl-glutaminase, protein-glutamine glutaminase, 6-aminohexanoate-dimer hydrolase, N-acetyldiaminopimelate deacetylase, acetylspermidine deacetylase, formamidase, pentanamidase, 4-acetamidobutyryl-CoA deacetylase, peptide-N(4)-(N-acetyl-beta-glucosaminyl)asparagine amidase, N-carbamoylputrescine amidase, allophanate hydrolase, long-chain-fatty-acyl-glutamate deacylase, N,N-dimethylformamidase, tryptophanamidase, N-benzyloxycarbonylglycine hydrolase, N-carbamoylsarcosine amidase, N-(long-chain-acyl)ethanolamine deacylase, mimosinase, acetylputrescine deacetylase, 4-acetamidobutyrate deacetylase, N(alpha)-benzyloxycarbonylleucine hydrolase, theanine hydrolase, 2-(hydroxymethyl)-3-(acetamidomethylene)succinate hydrolase, 4-methyleneglutaminase, N-formylglutamate deformylase, glycosphingolipid deacylase, aculeacin-A deacylase, N-feruloylglycine deacylase, D-benzoylarginine-4-nitroanilide amidase, carnitinamidase, chenodeoxycholoyltaurine hydrolase, urethanase, arylalkyl acylamidase, N-carbamoyl-D-amino acid hydrolase, glutathionylspennidine amidase, phthalyl amidase, N-acyl-D-amino-acid deacylase, N-acyl-D-glutamate deacylase, N-acyl-D-aspartate deacylase, biuret amidohydrolase, (S)—N-acetyl-1-phenylethylamine hydrolase, mandelamide amidase, N-carbamoyl-L-amino-acid hydrolase, peptide deformylase, N-acetylglucosaminyl-phosphatidylinositol deacetylase, adenosylcobinamide hydrolase, N-substituted formamide deformylase, pantetheine hydrolase, and glutaryl-7-aminocephalosporanic-acid acylase.

Additional amide hydrolases include, for example, cyclic amide hydrolases such as barbiturase, dihydropyrimidinase, dihydroorotase, carboxymethylhydantoinase, allantoinase, beta-lactamase, imidazolonepropionase, 5-oxoprolinase (ATP-hydrolyzing), creatininase, L-lysine-lactamase, 6-aminohexanoate-cyclic-dimer hydrolase, 2,5-dioxopiperazine hydrolase, N-methylhydantoinase (ATP-hydrolyzing), cyanuric acid amidohydrolase, maleimide hydrolase, and hydroxyisourate hydrolase.

Non-limiting examples of additional amide hydrolases include, but are not limited to, peptide hydrolases such as aminopeptidases (e.g., leucyl aminopeptidase, membrane alanyl aminopeptidase, cystinyl aminopeptidase, tripeptide aminopeptidase, prolyl aminopeptidase, aminopeptidase B, glutamyl aminopeptidase, Xaa-Pro aminopeptidase, bacterial leucyl aminopeptidase, clostridial aminopeptidase, cytosol alanyl aminopeptidase, aminopeptidase Y, Xaa-Trp aminopeptidase, tryptophanyl aminopeptidase, methionyl aminopeptidase, D-stereospecific aminopeptidase, aminopeptidase Ey, aspartyl aminopeptidase, aminopeptidase I, and PepB aminopeptidase), dipeptidases, dipeptidyl-peptidases, tripeptidyl-peptidases, peptidyl-dipeptidases, serine-type carboxypeptidases, metallocarboxypeptidases, cysteine-type carboxypeptidases, omega peptidases, serine endopeptidases, cysteine endopeptidases, aspartic endopeptidases, metalloendopeptidases, and threonine endopeptidases.

As used herein, the term “conjugate” refers to a chemical compound that has been formed by the joining or attachment of two or more compounds. In particular, a conjugate of the present invention comprises a substituted aminopyridine covalently attached to an organic molecule via an amide bond.

The term “organic molecule” is intended to include a compound that is usually composed of carbon atoms in rings or long chains, to which are attached other atoms of such elements as hydrogen, oxygen, and nitrogen. Examples of organic molecules include, but are not limited to, fatty acids, lipids, amino acids, peptides, polypeptides, proteins, glycoproteins, small organic molecules, polysaccharides, oligosaccharides, polynucleotides, oligonucleotides, fragments thereof, derivatives thereof, analogs thereof, etc. The organic molecule can be a naturally-occurring or synthetic compound.

The term “substrate” refers to a molecule upon which an enzyme acts. Enzymes catalyze chemical reactions involving the substrate. The substrate typically binds with the enzyme's active site, and an enzyme-substrate complex is formed. The substrate is then broken down into a product and is released from the active site. The active site is now free to accept another substrate molecule.

As used herein, the term “substrate for an amide hydrolase” refers to any conjugate of the present invention that can be cleaved at the amide bond formed by the covalent attachment of the substituted aminopyridine to the organic molecule.

A “fatty acid” is intended to include any of a large group of monobasic acids, especially those found in animal and vegetable fats and oils, having the general formula CₙH₂ₙ₊₁COOH. Fatty acids are typically saturated or unsaturated aliphatic compounds comprising an even number of carbon atoms. Examples of fatty acids include, but are not limited to, saturated fatty acids such as butyric acid (butanoic acid), caproic acid (hexanoic acid), caprylic acid (octanoic acid), capric acid (decanoic acid), lauric acid (dodecanoic acid), myristic acid (tetradecanoic acid), palmitic acid (hexadecanoic acid), stearic acid (octadecanoic acid), arachidic acid (eicosanoic acid), and behenic acid (docosanoic acid); and unsaturated fatty acids such as myristoleic acid, palmitoleic acid, oleic acid, linoleic acid, alpha-linoleic acid, arachidonic acid, eicosapentaenoic acid, erucic acid, and docosahexaenoic acid.

The term “amino acid” includes naturally-occurring α-amino acids and their stereoisomers, as well as unnatural amino acids and their stereoisomers. “Stereoisomers” of amino acids refers to mirror image isomers of the amino acids, such as L-amino acids or D-amino acids. For example, a stereoisomer of a naturally-occurring amino acid refers to the mirror image isomer of the naturally-occurring amino acid, i.e., the D-amino acid.

The terms “peptide,” “polypeptide,” and “protein” are used herein to refer to a polymer of amino acid residues. The terms apply to amino acid polymers in which one or more amino acid residue is an artificial chemical mimetic of a corresponding naturally-occurring amino acid, as well as to naturally-occurring amino acid polymers and non-naturally occurring amino acid polymers.

The term “substituted” refers to the replacement of an atom or a group of atoms of a compound with another atom or group of atoms. For example, an atom or a group of atoms may be substituted with one or more of the following substituents or groups: halo, nitro, C₁-C₈ alkyl, C₁-C₈ alkylamino, hydroxy C₁-C₈ alkyl, halo C₁-C₈ alkyl, carboxyl, hydroxyl, C₁-C₈ alkoxy, halo C₁-C₈ alkoxy, thio C₁-C₈ alkyl, aryl, aryloxy, C₃-C₈ cycloalkyl, C₁-C₈ alkyl, aryl, heteroaryl, aryl C₁-C₈ alkyl, heteroaryl C₁-C₈ alkyl, C₂-C₈ alkenyl containing 1 to 2 double bonds, C₂-C₈ alkynyl containing 1 to 2 triple bonds, C₂-C₈ alk(en)(yn)yl groups, cyano, formyl, C₁-C₈ alkylcarbonyl, arylcarbonyl heteroarylcarbonyl, carboxy, C₁-C₈ alkoxycarbonyl, aryloxycarbonyl, aminocarbonyl, C₁-C₈ alkylaminocarbonyl, C₁-C₈ dialkylaminocarbonyl, arylaminocarbonyl, diarylaminocarbonyl, aryl C₁-C₈ alkylaminocarbonyl, aryloxy, C₂-C₈ alkenyloxy, C₂-C₈ alkynyloxy, aryl C₁-C₈ alkoxy, amino C₁-C₈ alkyl, C₁-C₈ alkylamino, C₁-C₈ dialkylamino, arylamino C₁-C₈ alkyl, amino, arylamino, C₁-C₈ alkylarylamino, C₁-C₈ alkylcarbonylamino, arylcarbonylamino, azido, mercapto, C₁-C₈ alkylthio, arylthio, halo C₁-C₈ alkylthio, thiocyano, isothiocyano, C₁-C₈ alkylsulfinyl, C₁-C₈ alkylsulfonyl, arylsulfinyl, arylsulfonyl, aminosulfonyl, C₁-C₈ alkylaminosulfonyl, C₁-C₈ dialkylaminosulfonyl, and arylaminosulfonyl. When the term “substituted” appears prior to a list of possible substituted groups, it is intended that the term apply to every member of that group.

The term “unsubstituted” refers to a native compound that lacks replacement of an atom or a group of atoms.

As used herein, the term “alkyl” refers to a saturated hydrocarbon radical which may be straight-chain or branched-chain (e.g., ethyl, isopropyl, t-amyl, or 2,5-dimethylhexyl, etc.). This definition applies both when the term is used alone and when it is used as part of a compound term, such as “aralkyl,” “alkylamino,” and similar terms. In some embodiments, alkyl groups are those containing 1 to 24 carbon atoms. All numerical ranges in this specification and claims are intended to be inclusive of their upper and lower limits. Alkyl groups having a heteroatom (e.g., N, O, or S) in place of a carbon ring atom may be referred to as “heteroalkyl.” Additionally, the alkyl and heteroalkyl groups may be attached to other moieties at any position on the alkyl radical which would otherwise be occupied by a hydrogen atom (such as, for example, 2-pentyl, 2-methylpent-1-yl, and 2-propyloxy). The alkyl groups may also be optionally substituted with halogen atoms, or other groups such as oxo, cyano, nitro, alkyl, alkylamino, carboxyl, hydroxyl, alkoxy, aryloxy, and the like.

The terms “cycloalkyl” and “cycloalkenyl” refer to a saturated hydrocarbon ring and includes bicyclic and polycyclic rings. Similarly, cycloalkyl and cycloalkenyl groups having a heteroatom (e.g., N, O, or S) in place of a carbon ring atom may be referred to as “heterocycloalkyl”, “heterocyclyl,” and “heterocycloalkylene,” respectively. Examples of cycloalkyl and heterocyclyl groups include cyclohexyl, norbornyl, adamantyl, morpholinyl, thiomorpholinyl, dioxothiomorpholinyl, pyridinyl, and the like. The cycloalkyl and heterocycloalkyl moieties may also be optionally substituted with halogen atoms, or other groups such as nitro, alkyl, alkylamino, carboxyl, alkoxy, aryloxy and the like. In some embodiments, cycloalkyl and cycloalkenyl moieties are those having 3 to 12 carbon atoms in the ring (e.g., cyclohexyl, cyclooctyl, norbornyl, adamantyl, and the like). In some embodiments, heterocycloalkyl and heterocycloalkylene moieties are those having 1 to 3 hetero atoms in the ring (e.g., morpholinyl, thiomorpholinyl, dioxothiomorpholinyl, piperidinyl, and the like). Additionally, the term “(cycloalkyl)alkyl” refers to a group having a cycloalkyl moiety attached to an alkyl moiety. Examples are cyclohexylmethyl, cyclohexylethyl, and cyclopentylpropyl.

The term “alkenyl” as used herein refers to an alkyl group as described above which contains one or more sites of unsaturation that is a double bond. Similarly, the term “alkynyl” as used herein refers to an alkyl group as described above which contains one or more sites of unsaturation that is a triple bond.

The term “alkoxy” refers to an alkyl radical as described above which also bears an oxygen substituent which is capable of covalent attachment to another hydrocarbon radical (such as, for example, methoxy, ethoxy, aryloxy, and t-butoxy).

The term “aryl” refers to an aromatic carbocyclic substituent which may be a single ring or multiple rings which are fused together, linked covalently or linked to a common group such as an ethylene or methylene moiety. Similarly, aryl groups having a heteroatom (e.g., N, O, or S) in place of a carbon ring atom are referred to as “heteroaryl.” Examples of aryl and heteroaryl groups include phenyl, naphthyl, biphenyl, diphenylmethyl, 2,2-diphenyl-1-ethyl, thienyl, pyridyl, and quinoxalyl. The aryl and heteroaryl moieties may also be optionally substituted with halogen atoms, or other groups such as nitro, alkyl, alkylamino, carboxyl, alkoxy, phenoxy, and the like. Additionally, the aryl and heteroaryl groups may be attached to other moieties at any position on the aryl or heteroaryl radical which would otherwise be occupied by a hydrogen atom (such as, for example, 2-pyridyl, 3-pyridyl, and 4-pyridyl).

The terms “arylalkyl,” “arylalkenyl,” and “aryloxyalkyl” refer to an aryl radical attached directly to an alkyl group, an alkenyl group, or an oxygen which is attached to an alkyl group, respectively. For brevity, aryl as part of a combined term as above is meant to include heteroaryl as well.

The term “halo” or “halogen,” by itself or as part of another substituent, means, unless otherwise stated, a fluorine, chlorine, bromine, or iodine atom. Additionally, terms such as “haloalkyl” are meant to include monohaloalkyl and polyhaloalkyl. For example, the term “C₁-C₆ haloalkyl” is meant to include trifluoromethyl, 2,2,2-trifluoroethyl, 4-chlorobutyl, 3-bromopropyl, and the like.

The term “hetero” as used in a “heteroatom-containing alkyl group” (i.e., a “heteroalkyl” group) or a “heteroatom-containing aryl group” (i.e., a “heteroaryl” group) refers to a molecule, linkage, or substituent in which one or more carbon atoms are replaced with an atom other than carbon, e.g., nitrogen, oxygen, sulfur, phosphorus, or silicon. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

“Inhibitors,” “activators,” and “modulators” of activity are used herein to refer to inhibitory, activating, or modulating molecules, respectively, identified using in vitro and in vivo assays for activity, e.g., ligands, mimetics, agonists, antagonists, and their homologs and derivatives. The term “modulator” includes inhibitors and activators. Inhibitors are agents that, e.g., inhibit the enzymatic activity of an amide hydrolase. Inhibitors can also bind to, partially or totally block stimulation or activity, decrease, prevent, delay activation, inactivate, desensitize, or down-regulate the activity of an amide hydrolase. Activators are agents that, e.g., induce or activate the enzymatic activity of an amide hydrolase. Modulators include naturally-occurring and synthetic ligands, mimetics, antagonists, agonists, small chemical molecules, antibodies, inhibitory RNA molecules (e.g., siRNA or antisense RNA), and the like.

Assays to identify inhibitors and activators include, e.g., applying a putative modulator compound to a conjugate of the present invention in the presence of an amide hydrolase, and then determining the effect of the modulator compound on the ability of the amide hydrolase to liberate the substituted aminopyridine. Samples or assays comprising an amide hydrolase that are treated with a potential activator, inhibitor, or modulator can be compared to control samples without the inhibitor, activator, or modulator to examine the extent of the effect on amide hydrolase activity. Control samples (i.e., untreated with modulators) can be assigned a relative activity value of 100%. Inhibition can be achieved when the activity value of an amide hydrolase relative to the control is less than about 80%, 75%, 70%, 65%, 60%, 55%, 50%, 45%, 40%, 35%, 30%, 25%, 20%, 15%, 10%, or 5%. Activation can be achieved when the activity value of an amide hydrolase relative to the control is greater than about 110%, optionally greater than about 150% (e.g., greater than about 200-500%, 1000-3000%, etc.).

The term “test compound,” “drug candidate,” “modulator,” or grammatical equivalents as used herein describes any molecule, either naturally-occurring or synthetic, e.g., protein, polypeptide, peptide (e.g., from about 5-25 amino acids in length, from about 10-20 or about 12-18 amino acids in length, or about 12, 15, or 18 amino acids in length), small organic molecule, polysaccharide, lipid, fatty acid, polynucleotide, RNAi, antisense RNA, oligonucleotide, etc. The test compound can be in the form of a library of test compounds, such as a combinatorial or randomized library that provides a sufficient range of diversity. Conventionally, new chemical entities with useful properties are generated by identifying a test compound (called a “lead compound” with some desirable property or activity, e.g., stimulating or inhibiting activity, creating variants of the lead compound, and evaluating the property and activity of those variant compounds. Often, high-throughput screening (HTS) methods are employed for such an analysis.

By “therapeutically effective amount” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “subject” is defined herein to include animals such as mammals, including, but not limited to, primates (e.g., humans), cows, sheep, goats, horses, dogs, cats, rabbits, rats, mice, and the like. In some embodiments, the subject is a human.

### III. Description of the Embodiments

The present invention provides, inter alia, conjugates comprising a substituted aminopyridine covalently attached to an organic molecule via an amide bond. Such conjugates find utility as substrates for amide hydrolases, e.g., in assays for measuring amide hydrolase activity. The substituted aminopyridine conjugates described herein are also particularly useful for high-throughput screening of compounds that modulate (e.g., inhibit) the activity of amide hydrolases. Kits comprising the substituted aminopyridine conjugates of the present invention find utility in a wide range of applications including, for example, basic research, drug screening, and drug design.

A. Screening Methods

The present invention provides methods of identifying compounds that inhibit amide hydrolase activity, for example, by inhibiting the binding of an amide hydrolase to its substrate. The compounds find use in treating any of a variety of diseases or conditions associated with aberrant (e.g., increased or undesirable) amide hydrolase activity. As a non-limiting example, compounds that inhibit the activity of fatty acid amide hydrolase (FAAH) can be used to provide therapeutic benefits such as hypoalgesia, relief of pain and spasticity, relief of anxiety, and protection from inflammation. Another non-limiting example would be screening for inhibitors of bacterial amidases that degrade antibiotics to provide the therapeutic benefit of overcoming bacterial resistance to antibiotics.

Using the assays described herein, one can identify lead compounds that are suitable for further testing to identify those that are therapeutically effective modulating agents by screening a variety of compounds and mixtures of compounds for their ability to inhibit amide hydrolase activity. Compounds of interest can be either synthetic or naturally-occurring.

Screening assays can be carried out in vitro or in vivo. Typically, initial screening assays are carried out in vitro, and can be confirmed in vivo using cell-based assays or animal models. The screening methods are designed to screen large chemical or polymer libraries comprising, e.g., small organic molecules, peptides, peptidomimetics, peptoids, proteins, polypeptides, glycoproteins, oligosaccharides, or polynucleotides such as inhibitory RNA (e.g., siRNA, antisense RNA), by automating the assay steps and providing compounds from any convenient source to the assays, which are typically run in parallel (e.g., in microtiter formats on microtiter plates in robotic assays).

The present invention also provides in vitro assays in a high-throughput format. For each of the assay formats described, “no modulator” control reactions, which do not include a modulator, provide, e.g., a background level of amide hydrolase activity. In the high-throughput assays of the present invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) modulators. If 1536 well plates are used, then a single plate can easily assay from about 100 to about 1500 different compounds. It is possible to assay many different plates per day; assay screens for up to about 6000-20,000, and even up to about 100,000-1,000,000 different compounds is possible using the integrated systems of the present invention. The steps of labeling, addition of reagents, fluid changes, and detection are compatible with full automation, for instance, using programmable robotic systems or “integrated systems” commercially available, for example, through BioTX Automation (Conroe, Tex.), Qiagen (Valencia, Calif.), Beckman Coulter (Fullerton, Calif.), and Caliper Life Sciences (Hopkinton, Mass.).

Essentially, any chemical compound can be tested as a potential modulator of amide hydrolase activity for use in the methods of the present invention. Most preferred are generally compounds that can be dissolved in aqueous or organic solutions. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland), as well as providers of small organic molecule and peptide libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), and Tripos, Inc. (St. Louis, Mo.).

In some embodiments, modulators of amide hydrolase activity can be identified by screening a combinatorial library containing a large number of potential therapeutic compounds (potential modulator compounds). Such “combinatorial chemical or peptide libraries” can be screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

The preparation and screening of combinatorial chemical libraries is well known to those of skill in the art (see, e.g., Beeler et al., Curr Opin Chem. Biol., 9:277 (2005); and Shang et al., Curr Opin Chem. Biol., 9:248 (2005)). Libraries of use in the present invention can be composed of amino acid compounds, nucleic acid compounds, carbohydrates, or small organic compounds. Carbohydrate libraries have been described in, for example, Liang et al., Science, 274:1520-1522 (1996); and U.S. Pat. No. 5,593,853.

Representative amino acid compound libraries include, but are not limited to, peptide libraries (see, e.g., U.S. Pat. Nos. 5,010,175; 6,828,422; and 6,844,161; Furka, Int. J. Pept. Prot. Res., 37:487-493 (1991); Houghton et al., Nature, 354:84-88 (1991); and Eichler, Comb Chem High Throughput Screen., 8:135 (2005)), peptoids (PCT Publication No. WO 91/19735), encoded peptides (PCT Publication No. WO 93/20242), random bio-oligomers (PCT Publication No. WO 92/00091), vinylogous polypeptides (Hagihara et al., J. Amer. Chem. Soc., 114:6568 (1992)), nonpeptidal peptidomimetics with β-D-glucose scaffolding (Hirschmann et al., J. Amer. Chem. Soc., 114:9217-9218 (1992)), peptide nucleic acid libraries (see, e.g., U.S. Pat. No. 5,539,083), antibody libraries (see, e.g., U.S. Pat. Nos. 6,635,424 and 6,555,310; PCT Application No. PCT/US96/10287; and Vaughn et al., Nature Biotechnology, 14:309-314 (1996)), and peptidyl phosphonates (Campbell et al., J. Org. Chem., 59:658 (1994)).

Representative nucleic acid compound libraries include, but are not limited to, genomic DNA, cDNA, mRNA, inhibitory RNA (e.g., RNAi, siRNA), and antisense RNA libraries. See, e.g., Ausubel, Current Protocols in Molecular Biology, eds. 1987-2005, Wiley Interscience; and Sambrook and Russell, Molecular Cloning: A Laboratory Manual, 2000, Cold Spring Harbor Laboratory Press. Nucleic acid libraries are described in, for example, U.S. Pat. Nos. 6,706,477; 6,582,914; and 6,573,098. cDNA libraries are described in, for example, U.S. Pat. Nos. 6,846,655; 6,841,347; 6,828,098; 6,808,906; 6,623,965; and 6,509,175. RNA libraries, for example, ribozyme, RNA interference, or siRNA libraries, are described in, for example, Downward, Cell, 121:813 (2005) and Akashi et al., Nat. Rev. Mol. Cell Biol., 6:413 (2005). Antisense RNA libraries are described in, for example, U.S. Pat. Nos. 6,586,180 and 6,518,017.

Representative small organic molecule libraries include, but are not limited to, diversomers such as hydantoins, benzodiazepines, and dipeptides (Hobbs et al., Proc. Nat. Acad. Sci. USA, 90:6909-6913 (1993)); analogous organic syntheses of small compound libraries (Chen et al., J. Amer. Chem. Soc., 116:2661 (1994)); oligocarbamates (Cho et al., Science, 261:1303 (1993)); benzodiazepines (e.g., U.S. Pat. No. 5,288,514; and Baum, C&EN, January 18, page 33 (1993)); isoprenoids (e.g., U.S. Pat. No. 5,569,588); thiazolidinones and metathiazanones (e.g., U.S. Pat. No. 5,549,974); pyrrolidines (e.g., U.S. Pat. Nos. 5,525,735 and 5,519,134); morpholino compounds (e.g., U.S. Pat. No. 5,506,337); tetracyclic benzimidazoles (e.g., U.S. Pat. No. 6,515,122); dihydrobenzpyrans (e.g., U.S. Pat. No. 6,790,965); amines (e.g., U.S. Pat. No. 6,750,344); phenyl compounds (e.g., U.S. Pat. No. 6,740,712); azoles (e.g., U.S. Pat. No. 6,683,191); pyridine carboxamides or sulfonamides (e.g., U.S. Pat. No. 6,677,452); 2-aminobenzoxazoles (e.g., U.S. Pat. No. 6,660,858); isoindoles, isooxyindoles, or isooxyquinolines (e.g., U.S. Pat. No. 6,667,406); oxazolidinones (e.g., U.S. Pat. No. 6,562,844); and hydroxylamines (e.g., U.S. Pat. No. 6,541,276).

Devices for the preparation of combinatorial libraries are commercially available. See, e.g., 357 MPS and 390 MPS from Advanced Chem. Tech (Louisville, Ky.), Symphony from Rainin Instruments (Woburn, Mass.), 433A from Applied Biosystems (Foster City, Calif.), and 9050 Plus from Millipore (Bedford, Mass.).

B. Methods of Administration and Pharmaceutical Compositions

Molecules and compounds identified that modulate amide hydrolase activity can be administered via any of the routes described above. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003, supra).

Formulations suitable for oral administration can comprise: (a) liquid solutions, such as an effective amount of a modulator suspended in diluents, e.g., water, saline, or PEG 400; (b) capsules, sachets, or tablets, each containing a predetermined amount of a modulator, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise a modulator in a flavor, e.g., sucrose, as well as pastilles comprising the modulator in an inert base, such as gelatin and glycerin or sucrose and acacia emulsions, gels, and the like containing, in addition to the modulator, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of the present invention, compositions can be administered, for example, by intravenous infusion, orally, topically, intraperitoneally, intravesically, or intrathecally. Parenteral administration, oral administration, and intravenous administration are the preferred methods of administration. The formulations of compounds can be presented in unit-dose or multi-dose sealed containers, such as ampoules and vials.

Injection solutions and suspensions can be prepared from sterile powders, granules, and tablets of the kind previously described.

The pharmaceutical preparation is preferably in unit dosage form. In such form the preparation is subdivided into unit doses containing appropriate quantities of the active component, e.g., a modulator. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

In therapeutic use for the treatment of a disease or condition associated with increased or undesirable amide hydrolase activity, the compounds utilized in the pharmaceutical methods of the present invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. The dose administered to a patient, in the context of the present invention, should be sufficient to effect a beneficial therapeutic response in the patient over time. The size of the dose will also be determined by the existence, nature, and extent of any adverse side-effects that accompany the administration of a particular modulator in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

C. Therapeutic Applications

In certain aspects, the substituted aminopyridine conjugates of the present invention are particularly useful in assays for detecting aberrant amide hydrolase activity associated with a disease or disorder in a subject. Examples of diseases or disorders suitable for detection include, but are not limited to, allergy, autoimmune disease, behavioral disorder, birth defect, blood disorder, bone disease, cancer, tooth disease, depressive disorder, dissociative disorder, ear condition, eating disorder, eye condition, food allergy, food-borne illness, gastrointestinal disease, genetic disorder, heart disease, hormonal disorder, immune deficiency, infectious disease, inflammatory disease, insect-transmitted disease, nutritional disorder, kidney disease, leukodystrophy, liver disease, mental health disorder, metabolic disease, mood disorder, musculodegenerative disorder, neurological disorder, neurodegenerative disorder, neuromuscular disorder, personality disorder, phobia, pregnancy complication, prion disease, prostate disease, psychological disorder, psychiatric disorder, respiratory disease, sexual disorder, skin condition, sleep disorder, speech-language disorder, sports injury, tropical disease, vascular or circulatory disease, vestibular disorder, and wasting disease.

Cancer generally includes any of various malignant neoplasms characterized by the proliferation of anaplastic cells that tend to invade surrounding tissue and metastasize to new body sites. Non-limiting examples of different types of cancer include ovarian cancer, breast cancer, lung cancer, bladder cancer, thyroid cancer, liver cancer, pleural cancer, pancreatic cancer, cervical cancer, prostate cancer, testicular cancer, colon cancer, anal cancer, bile duct cancer, gastrointestinal carcinoid tumors, esophageal cancer, gall bladder cancer, rectal cancer, appendix cancer, small intestine cancer, stomach (gastric) cancer, renal cancer (i.e., renal cell carcinoma), cancer of the central nervous system, skin cancer, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

Inflammatory diseases typically include diseases or disorders characterized or caused by inflammation. Inflammation can result from a local response to cellular injury that is marked by capillary dilatation, leukocytic infiltration, redness, heat, and pain that serves as a mechanism initiating the elimination of noxious agents and damaged tissue. The site of inflammation can include, for example, the lungs, the pleura, a tendon, a lymph node or gland, the uvula, the vagina, the brain, the spinal cord, nasal and pharyngeal mucous membranes, a muscle, the skin, bone or bony tissue, a joint, the urinary bladder, the retina, the cervix of the uterus, the canthus, the intestinal tract, the vertebrae, the rectum, the anus, a bursa, a follicle, and the like. Examples of inflammatory diseases include, but are not limited to, inflammatory bowel disease (e.g., Crohn's disease or ulcerative colitis), rheumatoid diseases such as rheumatoid arthritis, fibrositis, pelvic inflammatory disease, acne, psoriasis, actinomycosis, dysentery, biliary cirrhosis, Lyme disease, heat rash, Stevens-Johnson syndrome, mumps, pemphigus vulgaris, and blastomycosis.

Autoimmune diseases generally include diseases or disorders resulting from an immune response against a self-tissue or tissue component such as, e.g., a self-antibody response or cell-mediated response. Examples of autoimmune diseases include, without limitation, organ-specific autoimmune diseases, in which an autoimmune response is directed against a single tissue, such as Type I diabetes mellitus, myasthenia gravis, vitiligo, Graves' disease, Hashimoto's disease, Addison's disease, autoimmune gastritis, and autoimmune hepatitis; and non-organ specific autoimmune diseases, in which an autoimmune response is directed against a component present in several or many organs throughout the body, such as systemic lupus erythematosus, progressive systemic sclerosis and variants, polymyositis, and dermatomyositis. Additional autoimmune diseases include, for example, pernicious anemia, primary biliary cirrhosis, autoimmune thrombocytopenia, Sjögren's syndrome, and multiple sclerosis.

In some embodiments, the substituted aminopyridine conjugates of the present invention are useful for detecting aberrant amide hydrolase activity associated with a neurological or musculoskeletal disorder. Examples of such disorders include, but are not limited to, Aicardi syndrome, Alzheimer's disease, amnesia, amyotrophic lateral sclerosis (Lou Gehrig's Disease), anencephaly, anxiety disorder, aphasia, arachnoiditis, Arnold Chiari malformation, ataxia telangiectasia, Batten disease, Bell's palsy, brachial plexus injury, brain injury, brain tumor, Charcol-Marie-Tooth disease, chronic pain, encephalitis, epilepsy, essential tremor, Guillain-Barre Syndrome, hydrocephalus, hyperhidrosis, Krabbes disease, meningitis, Moebius syndrome, muscular dystrophy, multiple sclerosis, Parkinson's disease, peripheral neuropathy, postural or orthostatic tachycardia syndrome, progressive supranuclear palsy, Reye's syndrome, shingles, Shy-Drager Syndrome, spasmodic torticollis, spina bifida, spinal muscular atrophy, Stiff Man syndrome, synesthesia, syringomyelia, thoracic outlet syndrome, Tourette syndrome, toxoplasmosis, and trigeminal neuralgia.

In other embodiments, the substituted aminopyridine conjugates described herein are useful in assays for detecting aberrant amide hydrolase activity associated with a vascular or circulatory disease. Non-limiting examples of such diseases include elephantiasis, hemochromatosis, hemophilia, hypertension, hypotension, Klippel-Trenaunay-Weber syndrome, lymphedema, neutropenia, peripheral vascular disease (PVD), phlebitis, Raynaud's phenomenon, thrombosis, twin-to-twin transfusion syndrome, or vasculitis. Other vascular or circulatory diseases include heart diseases such as, for example, arrhythmogenic right ventricular dysplasia, atherosclerosis/arteriosclerosis, cardiomyopathy, congenital heart disease, endocarditis, enlarged heart, heart attack, heart failure, heart murmur, heart palpitations, high cholesterol, high tryglycerides, hypertension, long QT syndrome, mitral valve prolapse, postural orthostatic tachycardia syndrome, tetralogy of fallots, and thrombosis.

In further embodiments, the substituted aminopyridine conjugates of the present invention are useful for detecting aberrant amide hydrolase activity associated with a liver disease including, but not limited to, alpha-1 antitrypsin deficiency, chronic liver disease, cirrhosis, fatty liver and non-alcoholic steatohepatitis, Gilbert's syndrome, hepatitis (hepatitis A, B, or C), liver cancer, and polycystic liver disease.

D. Kits of the Invention

The present invention also provides kits to facilitate and/or standardize the use of the compositions provided herein, as well as to facilitate the methods described herein. Materials and reagents to carry out these various methods can be provided in kits to facilitate execution of the methods. As used herein, the term “kit” includes a combination of articles that facilitates a process, assay, analysis, or manipulation. In particular, kits comprising the substituted aminopyridine conjugates of the present invention find utility in a wide range of applications including, for example, basic research, drug screening, and drug design.

Kits can contain chemical reagents (e.g., substituted aminopyridine conjugates, amide hydrolases, etc.) as well as other components. In addition, the kits of the present invention can include, without limitation, instructions to the kit user (e.g., directions for use of the conjugate in determining amide hydrolase activity, directions for use of the conjugate in identifying a compound that inhibits an amide hydrolase, etc.), apparatus and/or reagents for measuring the fluorescence of free substituted aminopyridines, apparatus and/or reagents for performing low-, medium-, or high-throughput screening assays for modulators of amide hydrolase activity, reagents for bacterial cell transformation, reagents for eukaryotic cell transfection, previously transformed or transfected host cells, sample tubes, holders, trays, racks, dishes, plates, solutions, buffers or other chemical reagents, suitable samples to be used for standardization, normalization, and/or control samples. Kits of the present invention can also be packaged for convenient storage and safe shipping, for example, in a box having a lid.

### IV. Examples

The following examples are offered to illustrate, but not to limit, the claimed invention.

### Example 1

### Highly Sensitive Fluorescent Assays for Fatty Acid Amide Hydrolase

This example describes the development of novel and highly sensitive fluorescent substrates for fatty acid amide hydrolase (FAAH) that are based on substituted aminopyridines. In particular, an examination of the relationship between the structure and fluorescence of substituted aminopyridines indicated that a methoxy group para to the amino group in the pyridine ring greatly increased the fluorescence of substituted aminopyridines (i.e., quantum yields approached one unity). These novel fluorescent reporters had high Stokes' shifts of 94 nm, and their fluorescence in buffer systems increased with pH values from neutral to basic. In addition, fluorescent substrates with these reporters displayed very low fluorescent background and high aqueous solubility. Most importantly, fluorescent assays for FAAH based on these substrates were at least 25 times more sensitive than related compounds with either colorimetric or fluorescent reporters published in the literature, which would advantageously result in shortening the assay time and decreasing the amount of protein used in the assay. Such sensitive assays facilitate identifying FAAH inhibitors and distinguishing between their relative potency. Thus, the fluorescent FAAH substrates described herein provide a valuable tool for use in assays of FAAH activity and to screen for FAAH inhibitors by high throughput assays instead of using costly and labor-intensive radioactive ligands.

### Results and Discussion

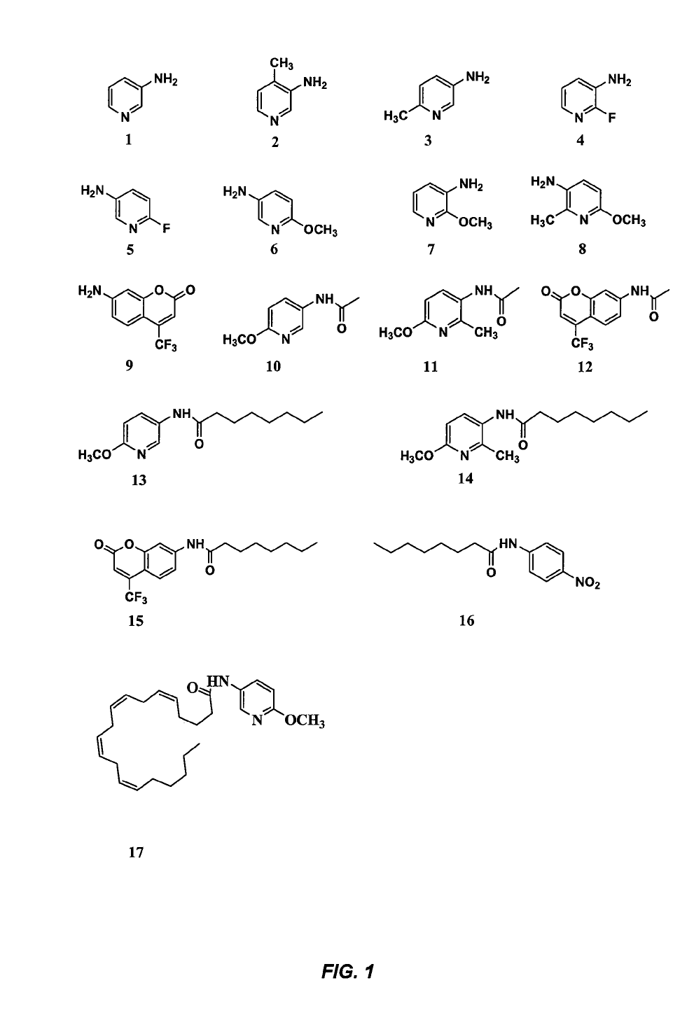

Optical properties. The molar extinction coefficient for absorption, fluorescence spectral profiles, and quantum yield of a fluorophore are the most important indexes for determining its fluorescent intensity in the enzymatic reaction media. Due to deprotonization or protonization, aminopyridines (e.g., 2-aminopyridine) can display from over one unit (1.20) to almost zero (0.05) of quantum yields in the different aqueous media (Rusakowicz et al., J. Phys. Chem., 72:2680-2681 (1968)). Neutral water with 1% ethanol (dissolving the aminopyridines) was chosen as a medium for measuring the indexes described above. Dependence of fluorescent intensity on pH values was shown next. As shown in Table 1, the results with neutral water as a medium indicated that these indexes varied greatly with the substituted group and position on the 3- or 5-aminopyridine. For example, compound (7) displayed a higher molar extinction coefficient, but much lower quantum yield than compound (6), which contains the same methoxy group, but at a different position (FIG. 1). Compounds (6) and (8) possessed almost one unity of quantum yields (for efficient laser dyes it approaches unity), and also possessed red-shift excitation and emission wavelength when compared to other aminopyridines. However, the Stokes' shift for all aminopyridines examined was almost the same. These interesting results provided the impetus for further investigating their application as fluorescent reporters of enzymatic activities.

TABLE 1
Optical properties of different substituted aminopyridines.

| Compound No. | εₘₐₓ (M⁻¹cm⁻¹)^{a} | Ex (nm)^{b} | Em (nm)^{b} | Ø_{F}^{c} |
|---|---|---|---|---|
| 1 | 2890 (2800)^{d} | 286 | 378 | 0.32^{d} |
| 2 | 2580 | 280 | 376 | 0.53 |
| 3 | 2700 | 290 | 384 | 0.62 |
| 4 | 3670 | 282 | 374 | 0.04 |
| 5 | 2360 | 292 | 384 | 0.72 |
| 6 | 2340 | 302 | 396 | 0.95 |
| 7 | 4670 | 284 | 378 | 0.27 |
| 8 | 4130 | 304 | 392 | 0.97 |

^{a}Molar extinction coefficients were determined in H₂O containing 1% ethanol at a wavelength 290 nm and 30° C., and the final concentrations of the solute were 10, 20, 30, 40, or 50 μM.
^{b}Ex and Em were obtained from scanning from 250 to 600 nm and 360 to 600 nm by a 2 nm interval at 30° C., respectively, and 50 μM final concentration for each reporter in 0.1 M phosphate buffer. The standard deviation for each datum was ±2 nm.
^{c}Ø_{F:} standard deviations were under ±5%.
^{d}Data obtained from Weisstuch et al., J. Phys. Chem., 72: 1982-1987 (1968).

The dependence of relative fluorescent intensity on pH values. It is well-known that hydroxide ions are a fluorescent quencher for aminopyridines and α- or β-naphthylamine (Rusakowicz et al., supra; Boaz et al., J. Am. Chem. Soc., 72:3435-3443 (1950)). This effect dramatically decreases in fluorescent intensity at maximal emission wavelength or quantum yields of aminopyridines from acidic media to basic media. For example, the quantum yield of 3-aminopyridine drops from 1.07 in 0.1 NH₂SO₄ to 0.32 in water, or to 0.03 in 1 N NaOH (Rusakowicz et al., supra). This may be one of the major reasons that limit the application of aminopyridines as fluorescent reporters of enzymatic activities.

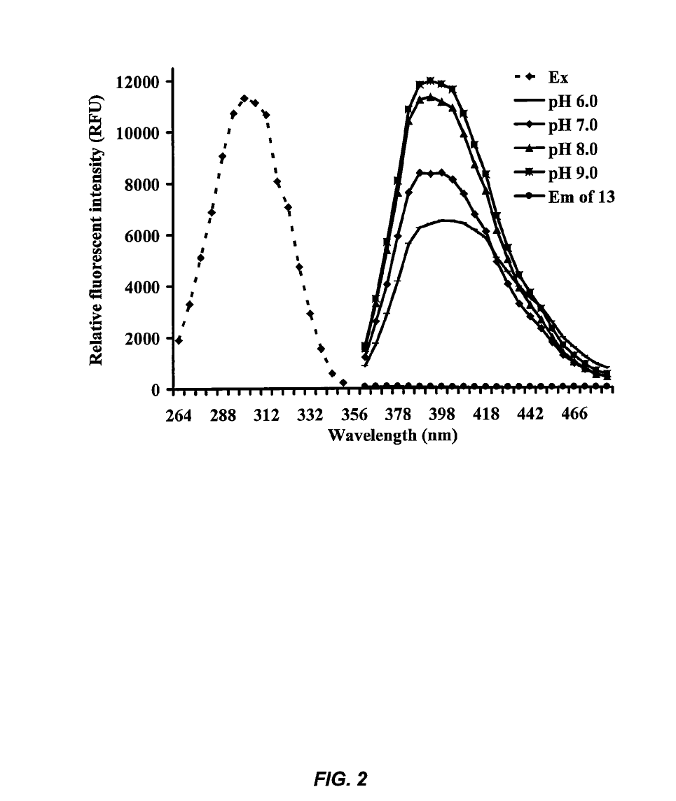

To determine whether hydroxide ions are also a fluorescent quencher of simple substituted aminopyridines, the dependence of relative fluorescent intensity on pH values in sodium phosphate buffer (pH 6.0-8.0) and Tris/HCl buffer (pH 9.0) was investigated. Results with 5-amino-2-methoxypyridine (6) as an example (FIG. 2) indicated that 1) there was no red shift for both either excitation or emission wavelength with an increase of pH from 6.0 to 9.0; 2) relative fluorescent intensity of either excitation (only showing pH 8.0 for clarity) or emission increased with an increase of pH value; 3) the relative fluorescent intensity of substrate (13) emission at the maximal excitation wavelength of (6) was very low (i.e., less than 200 RFU); and 4) the profiles of excitation and emission wavelengths were completely separated. Similar properties were observed with compound (8). This demonstrates that the hydroxide ions in a buffer system do not quench the fluorescence of (6) as strongly as they do for compound (1). This information can be used in several ways to reduce false positives in a high-throughput screen. In one embodiment, the sample could be run in duplicate and the reaction ended in one case with acid resulting in an increase in fluorescence and with base in the other case resulting in a decrease in fluorescence. Alternatively, for positive results (i.e., inhibition), the reaction could be ended with acidic hexanol or other solvent. This will cause the substituted aminopyridine to increase fluorescence and remain in the aqueous phase and potentially interfering materials to move to the upper phase, which can be interrogated separately in plate readers. Hexanol will not damage ELISA plates, but a variety of other solvents can be used, for example, to move the substituted aminopyridine from the hyperphase to the hypophase.

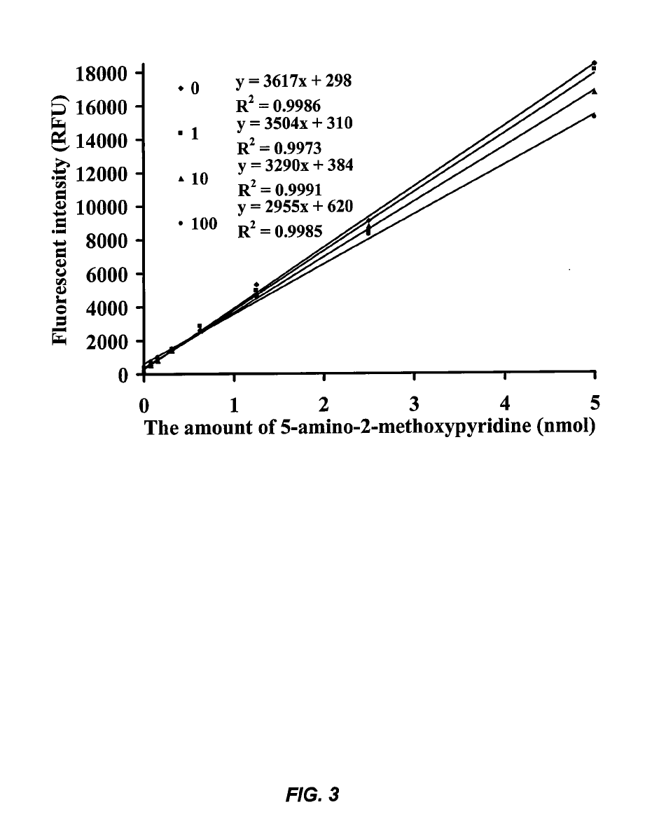

The effects of protein amount on the relative fluorescent intensity. In addition to the selectivity of enzymes toward their substrates, there are at least two other factors that affect the sensitivity of assays. First, it is a common phenomenon or inevitable that proteins quench the fluorescence of reporters. The results shown in FIG. 3 suggest that bovine serum albumin (BSA) quenched the fluorescence of compound (6), and the strength of quenching was dependent on the amount of BSA used. Similar phenomena were observed with compounds (8) and (9). Second, some ingredients in proteins may produce a signal at the maximal excitation wavelength of the reporter, especially for low wavelength reporters such as aminopyridines, resulting in lower sensitivity. For example, at the maximal excitation wavelength, blank noise from BSA at 10 μg per well (200 μl) was less than 200 RFU, and increased to 400 RFU when BSA was at 100 μg per well. Under the same conditions, blank noise from the compound (9) was less than 100 RFU at 100 μg per well. It seems that high protein (e.g., BSA) concentrations in assays typically produce higher background noise for aminopyridines than 7-amino-4-trifluorocoumarin (9), which is one of the factors determining the sensitivity of assays. However, the final protein concentration in high-throughput format systems is usually less than 10 μg per well; thus, this background noise will have little affect on the sensitivity of aminopyridines.

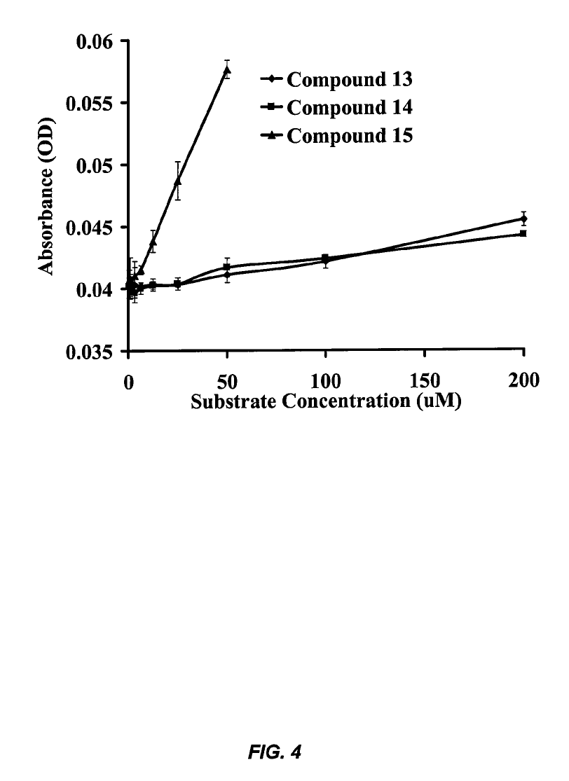

Comparison of aqueous solubility of substrates. Substrate solubility in aqueous media is one of the important components when one considers a kinetic study. Although this may be easily solved by adding more co-solvent or detergents, some of the activities may be sacrificed or enzyme properties altered. Based on the catalytic properties displayed by FAAH for the panel of p-nitroaniline substrates, the length of acyl chain (C6-C9) displayed a relatively strong enzyme-substrate interaction (Patricelli et al., Biochem., 40:6107-6115 (2001)). Thus, the C8 compound was chosen for comparison of substrates with p-nitroaniline, 7-amino-4-trifluoromethylcoumarin (9), and aminopyridines. With the same acid moiety, comparison of the aqueous solubility of pyridine substrates (e.g., 13, 14) to a substrate with 7-amino-4-trifluoromethylcoumarin (15) (FIG. 4) suggested that pyridine substrates have much higher solubility in 0.1 M phosphate buffer (pH 8.0) than the substrate (e.g., 15) with a coumarin group. This may partially contribute to the high substrate selectivity of FAAH described below.

Comparison of the lowest detection limits of microsomal FAAH with different substrates. As described above, protein quenching and background noises resulting from some ingredients in proteins are possibly the two factors that may determine the sensitivity of assays. Aminopyridines typically have a high molar extinction coefficient and quantum yields. However, another major factor that determines the sensitivity of assays when measuring hydrolytic activities results from the selectivity of hydrolases toward their substrates. For high throughput assays, it is the ability to detect the lowest possible enzyme concentration with acceptable signal to noise that determines practical sensitivity. The lowest detection limit for detecting FAAH with a particular substrate was determined from a signal at 10 min after a kinetic measurement that was equal to three times the background noise or base line. As shown in Table 2, the results indicated that 1) background noise or base line varied with the substrates, with higher background observed for (13) and (14) than (15) and (17), which may have resulted from a higher substrate concentration of (13) and (14) (i.e., 200 μM) as compared to (15) and (17) (i.e., 50 μM); 2) the lowest detection limits of proteins for substrates (13), (14), and (17) (0.55, 3.3, and 2.5 μg protein/ml, respectively) were determined, but not determinable for substrates (15) and (16) even at 500 μg protein/ml in a buffer system with 10% DMSO and 0.1% Triton-X 100; and 3) the structure of the reporters in the substrates had a dramatic effect on the sensitivity of assays for this amidase. For example, substrate (15) with 7-amino-4-trifluoromethylcoumarin (9) was at least 900 times less sensitive than substrate (13) with a 5-amino-2-methoxypyridine (6) reporter, and 150 times less sensitive than substrate (14) with a 5-amino-2-methoxy-6-methylpyridine (8) reporter group.

TABLE 2
Comparison of the lowest detection limits of micrsomal FAAH toward the colorimetric
substrate (16) and fluorescent substrates (13), (14), (15), and (17)^{a}.

| | Protein | | Signal | Average | |
|---|---|---|---|---|---|
| Substrate | (μg/200 μ′) | | (OD or RFU) | (OD or RFU) | St Dev |
| (13) | 0.11 | base line | 310 290 296 314 | 302 | 11 |
| | | signal | 1083 1126 1069 1086 | 1091* | 24 |
| (14) | 0.67 | base line | 215 161 199 201 | 194 | 23 |
| | | signal | 760 697 694 760 | 728* | 38 |
| (15) | 100 | base line | 134 130 124 139 | 132 | 6 |
| | | signal | 154 150 157 182 | 161 | 15 |
| (16) | 100 | base line | 0.15 0.16 0.16 0.16 | 0.16 | 0.01 |
| | | signal | 0.16 0.17 0.18 0.19 | 0.18 | 0.01 |
| (17) | 0.5 | base line | 140 146 164 140 | 148 | 11 |
| | | signal | 566 590 534 545 | 559* | 24 |

^{a}Assays were performed in a total volume of 201 μl containing 0.1 M sodium phosphate buffer (pH 8.0) with 1% glycerol and 0.1% Triton X-100 (190 μl), diluted protein solution (10 μl), and 1 μl substrate in ethanol (final concentration 200 μM for (13) and (14)) or 1 μl substrate solution in DMSO (final concentration 25 μM for (15) and (16); 50 μM for (17)) at 37° C. Absorbance wavelength for (16) was 382 nm. Excitation and emission wavelengths for (13) and (17) were 302 and 396, for (14) were 304 and 392, and for (15) were 366 and 496, respectively.
*Asterisks indicate that the datum was equal to or over three times the corresponding base line.

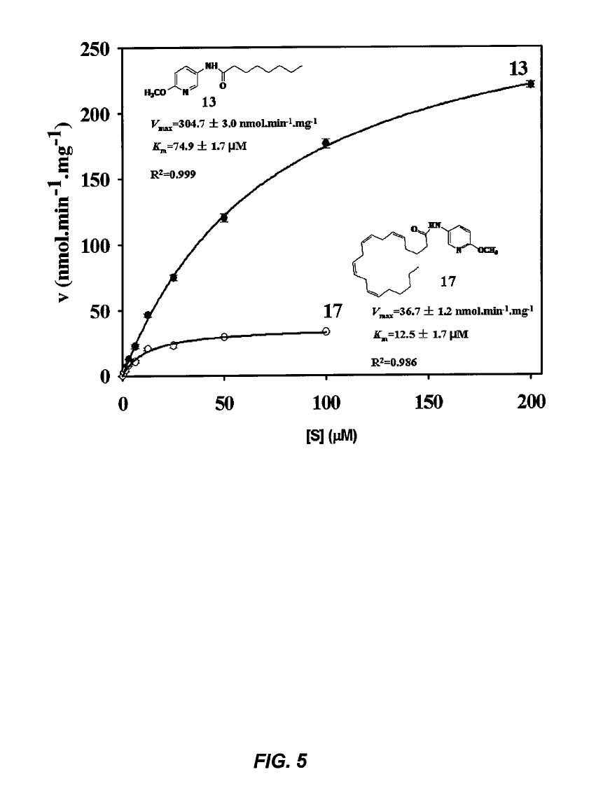

Kinetic data. To further compare substrate selectivity with other substrates published in the literature, a kinetic study with substrates (13) and (17) was conducted. As shown in FIG. 5 and Table 3, the results indicated that there was an 8-fold difference between (13) and (17) in Vₘₐₓ under conditions of saturating substrate concentration, similar to the results (5 times) obtained from the experiment involving the limit of protein detection (Table 2). The substrate selectivity of (17) was a little higher than that of (13) (4.1 and 3.0 for substrates (13) and (17), respectively). Although there is a difference in the percentage of FAAH in microsomes prepared from different laboratories, this difference was negligible when compared with the substrate selectivity published in the literature (Table 3). Amazingly, the substrate selectivity of FAAH toward fluorescent substrates with a substituted aminopyridine as a reporter (e.g., (13) and (17)) was over 50 times higher than that of a substrate used in colorimetric assays (e.g., oleamide; Table 3), or at least 25 times higher than that of a fluorescent substrate with coumarin as a reporter (e.g., AAMCA; Table 3).

TABLE 3
Comparison of kinetic data of FAAH toward different substrates.

| | | Vₘₐₓ (nmol · | | Selectivity |
|---|---|---|---|---|
| Substrates | Kₘ (μM) | min⁻¹ · mg⁻¹) | Vₘₐₓ/Kₘ | ratio^{c} |
| (13) | 74.9 | 305 | 4.1 | 74.5 |
| (17) | 12.5 | 36.7 | 3.0 | 54.5 |
| AAMCA^{a} | 0.48 | 5.8 × 10⁻² | 1.2 × 10⁻¹ | 2.2 |
| Oleamide^{b} | 104 | 5.7 | 5.5 × 10⁻² | 1.0 |

^{a}Data obtained from Ramarao et al., Anal. Biochem., 343: 143-151 (2005).
^{b}Data obtained from De Bank et al., Anal. Biochem., 69: 1187-1193 (2005).
^{c}Selectivity ratio was calculated by Vₘₐₓ/Kₘ of each substrate over that of oleamide (5.5 × 10⁻²).

This striking difference in sensitivity and substrate selectivity may result from a combination of multiple factors such as a higher aqueous solubility of the substrates, smaller reporter groups, and the nitrogen in the pyridine ring working as an electron donor or contributing to hydrogen bonding. For example, the only difference between substrate (14) with a 5-amino-2-methoxy-6-methylpyridine (8) reporter and substrate (13) with a 5-amino-2-methoxypyridine (6) reporter is one methyl group. The methyl group on the 2 position of the pyridine ring makes the electron richer for the nitrogen in the pyridine ring. It also, at least partially, blocks the nitrogen as an electron donor or in hydrogen bonding, which may facilitate hydrolysis by FAAH. This facilitation is supported by the fact that: 1) the sensitivity of FAAH toward substrate (14) is 6 times lower than that toward substrate (13); and 2) the substrate selectivity of FAAH toward oleamide is over 50 times lower than that of FAAH toward substrates (13) and (17) (Table 3). In addition, the difference in sensitivity between substrates (13) and (15) (i.e., at least 900 times difference; Table 2) demonstrates that an increase in the structural size of the reporter group also has an effect on substrate selectivity.

Application of the substrates. Extracts of the vehicle cells (High Five cells), cells infected by FAAH-expressing virus, human liver S9, and microsomes were chosen for FAAH enzyme-substrate analysis. As shown in Table 4, the results indicated that there were no detectable activities for substrates (11) and (15). Compared with the activities of microsomal enzymes from High Five cells, activities of microsomal enzymes from FAAH-expressing cells toward substrates (13), (14), and (17) were enhanced by 254, 31, and 4180 times, respectively. These results indicated that substrate (17) is more selective for FAAH than substrates (13) and (14). Similar phenomena were observed with human liver S9. Interestingly, there is another amide hydrolase that hydrolyzes long-chain fatty acid amides such as (13) and (14) in human liver microsomes because FAAH preferred substrate (13) to (14) (at least 6 times in Table 2) from both the sensitivity assay described above and specific activity (10 times in Table 4), but human liver microsomes preferred substrate (14) to (13) (at least 15 times in Table 4). This interesting result may facilitate purification and biological studies on the one or more amide hydrolases present in human hepatic microsomes.

TABLE 4
Specific activities of amidases toward fluorescent substrates^{a}.

| | Microsomes from | Microsomes from | | |
|---|---|---|---|---|
| | High Five cells | FAAH cells | Human liver S9 | |
| | (pmol/min/mg | (nmol/min/mg | (nmol/min/mg | Human liver microsomes |
| Substrate | protein) | protein) | protein) | (nmol/min/mg protein) |
| (10) | 56 ± 2 | NM^{b} | NM | NM |
| (11) | NM | NM | NM | NM |
| (12) | NM | NM | 0.18 ± 0.02 | 0.66 ± 0.06* |
| (13) | 793 ± 38.9 | 202.6 ± 3.3* | NM | 0.46 ± 0.06* |
| (14) | 591 ± 18.4 | 19.0 ± 0.5* | 0.42 ± 0.01 | 6.90 ± 0.25* |
| (15) | NM | NM | NM | NM |
| (17) | 5.62 ± 0.67 | 23.5 ± 0.7* | ND^{c} | ND |

^{a}Assays were performed in a total volume of 201 μl containing 0.1 M sodium phosphate buffer (pH 8.0) with 1% glycerol and 0.1% Triton X-100 (190 μl), diluted protein solution (10 μl), and 1 μl substrate in ethanol (final concentration 200 μM for (11), (12), (13), and (14)) or 1 μl substrate solution in DMSO (final concentration 25 μM for (15), 50 μM for (17)) at 37° C. Excitation and emission wavelengths for (10), (13), and (17) were 302 and 396, for (11) and (14) were 304 and 392, and for (12) and (15) were 366 and 496, respectively.
^{b}NM means “not measurable at the same protein concentration as other substrates.”
^{c}ND means “not determined.”
*Asterisks indicate that the datum was significantly enhanced from High Five cells to FAAH cells or from human liver S9 to human microsomes. Analysis was performed by Student's T-test (p ≦ 0.05).

Potential application of novel fluorescent reporters. Substituted aminopyidines display high fluorescence, and they represent a novel and structurally different class of fluorescent reporters from aminocoumarins. In fact, they possess many advantages over aminocoumarins such as higher fluorescence, better aqueous solubility, and smaller size. In addition, as described above for FAAH, the nitrogen in the pyridine ring works as an electron donor or contributes to hydrogen bonding and thus facilitate hydrolysis of substrates by amide hydrolases. They are potentially useful for all enzymes that hydrolyze amide bonds. These enzymes include, but are not limit to, fatty acid amide hydrolase (FAAH), nucleotide pyrophosphatase, transferases, hyaluronidase, numerous aminopeptidases, and the like.

Summary. The relationship between the structure of substituted aminopyridines and fluorescence was examined in this example. The results described herein indicate that the substituted groups on the pyridine ring dramatically affect the fluorescence quantum yields of aminopyridines. In particular, a methoxy group para to the amino group in the aminopyridines greatly increased fluorescence quantum yields. Based on substrate selectivity, novel fluorescent assays using substrates with aminopyridines as reporters were at least 50 times better than those assays (either fluorescent or colorimetric assays) published in the literature. Moreover, using the novel fluorescent substrates of the present invention, at least one other amide hydrolase in addition to FAAH in human liver microsomes was identified that could hydrolyze long chain fatty acid amides.

### Materials and Methods

Chemicals and enzymes. Fluorescent reporters including 3-amino-4-methylpyridine (2), 3-amino-6-methylpyridine (3), 3-amino-2-fluoropyridine (4), 5-amino-2-fluoropyridine (5), 5-amino-2-methoxypyridine (6), 3-amino-2-methoxypyridine (7), and 3-amino-6-methoxy-2-picoline (8) were purchased from Lancaster Synthesis, Inc. (2; Windham, N.H.), AB Chem., Inc. (3; Quebec, Canada), Matrix Scientific (4; Columbia, S.C.), Beta Pharma, Inc. (5; New Haven, Conn.), and Asychem (6-8; Durham, N.C.). 3-aminopyridine (1) and 7-amino-4-trifluoromethylcoumarin (9) were purchased from Sigma-Aldrich (St. Louis, Mo.). Pooled human liver S9 and microsomes were purchased from BD Biosciences (San Jose, Calif.).

Structural identification was based on data from proton nuclear magnetic resonance ('H-NMR) and gas chromatography/mass spectrometry (GC/MS). Proton NMR spectra were acquired from a Mercury 300 spectrometer (Varian Medical Systems, Inc.; Palo Alto, Calif.). Chemical shift values are given in parts per million (ppm) downfield from the internal standard (trimethylsilane). Signal multiplicities are represented as singlet (s), doublet (d), double doublet (dd), triplet (t), quartet (q), quintet (quint), multiplet (m), broad (br), and broad singlet (brs). Chemical purity of the final products was supported by the spectra described above, a single spot on TLC under a wavelength of 254 nm, and lack of fluorescence from 5-amino-2-methoxypyridine, 5-amino-2-methoxy-6-methylpyridine, or 7-amino-4-trifluoromethylcoumarin on TLC at a wavelength of 254 or 360 nm. Melting points were determined on an OptiMelt Automated Melting Point System (Stanford Research Systems; Sunnyvale, Calif.). GC/MS data were acquired on a Hewlett-Packard Model 5890 equipped with a HP 5973 mass spectral detector (Agilent Corp.; Arodale, Pa.) and a 30 m×0.25 mm i.d. capillary column coated with a 0.25 μm film of 5:95 methylphenyl-substituted dimethylpolysiloxane (DB-5 MS) (J & W Scientific; Folson, Calif.). The DB-5 MS column was carried out at 80° C. for 1 min, ramped to 300° C. (at an 11° C./min increment), and held for 5 min at this temperature. The injector port was operated in the splitless mode at 250° C., and helium was used as carrier gas at 0.8 mL/min. The mass spectral detector was set on full scan mode (m/z 50-550). Chemical purity was calculated by the relative peak area of the total current. An IR 100 spectrometer was internally built with easy-to-use Encompass™ software from Thermo Electron Corporation (San Jose, Calif.). Mass spectra were measured by LC-MS/MS (Waters 2790) using positive mode electrospray ionization.

Preparation of FAAH substrates (10-16). To an ice-cooled solution containing 100 mg of a fluorescent reporter such as a substituted aminopyridine or 7-amino-4-trifluoromethylcoumarin and 10 ml of dry CH₂Cl₂, the appropriate acid chloride (1.05 eqv) and 4-methylmorpholine (1.05 eqv) were slowly added. The reaction was warmed slowly to room temperature and stirred overnight. The reaction mixture was washed with saturated sodium bicarbonate solution and extracted three times with ethyl acetate (3×20 ml). The combined organic phase was dried by magnesium sulfate. A crude solid product was obtained after filtration and evaporation under reduced pressure. The crude product was chromatographed on a gradient of hexane and ethyl acetate.

N-(6-methoxypyridin-3-yl) acetamide (10): white needle crystal (49 mg, 37% yield). Melting point: 99.2-99.9° C. UVₘₐₓ (nm):278. ¹H NMR (CDCl₃): 8.11 (d, J=2.70, 1H, pyridine), 7.88 (dd, J=2.70, J₂=9.00, 1H pyridine), 7.41 (s, 1H, CONH), 6.70 (d, J=9.00, 1H pyridine), 3.90 (s, 3H, OCH₃), 2.16 (s, 3H, CH₃). ¹C NMR (CDCl₃): 169, 161, 139, 133, 129, 111, 54, 24; GC/MS EI m/z: 166 (M⁺, 76%), 124 (100%), 95 (63%). IR (KBr) νₘₐₓ, (cm⁻¹): 3238 (s, NH), 3062, 1652 (s, C═O), 1491, 1376, 1268, 1019, 843. LC-MS (ESI) m/z calculated for C₈H₁₀N₂O₂ [M+H]⁺ 167.07, found [M+H]⁺ 167.05.

N-(6-methoxy-2-methylpyridin-3-yl) acetamide (11): white solid (77 mg, 75% yield). Melting point: 130.1-130.4° C. UVₘₐₓ (nm): 276. ¹H NMR (CDCl₃): 7.74 (d, J=9.00, 1H, pyridine), 6.83 (s, 1H, CONH), 6.57 (d, J=9.00, 1H pyridine), 3.89 (s, 3H, OCH₃), 2.38 (s, 3H, CH₃), 2.19 (s, 3H, CH₃). ¹C NMR (CDCl₃): 169, 159, 149, 129, 115, 107, 54, 24, 21; GC/MS EI m/z: 180 (M⁺, 73%), 137 (100%), 109 (54%). IR (KBr) νₘₐₓ (cm⁻¹): 3266 (s, NH), 3120, 3037, 2949, 1652 (s, C═O), 1596, 1525, 1426, 1289, 1256, 1033, 825. LC-MS (ESI) m/z calculated for C₉H₁₂N₂O₂ [M+H]⁺ 181.09, found [M+H]⁺ 181.07.

N-(4-(trifluoromethyl)-2-oxo-2H-chromen-7-yl) acetamide (12): white solid (26 mg, 22% yield). Melting point: 109.3-111.4° C. UVₘₐₓ (nm):392. ¹H NMR (CDCl₃): 7.84 (dd, J=1.80, J₂=8.10, 1H, Ar), 7.20 (d, J=2.10, 1H, Ar), 7.17 (d, J=2.10, 1H, Ar), 6.88 (s, 1H, CH), 2.34 (s, 3H, CH₃). ¹C NMR (CDCl₃): 169, 161, 155, 149, 136, 125, 122, 118, 117, 108. GC/MS EI m/z: 271 (M⁺, 25%), 229 (91%), 201 (100%), 172 (26%). IR (KBr) νₘₐₓ (cm⁻¹): 3309 (s, NH), 3103, 1728 (s, C═O), 1612 (s, C═O), 1471, 1373, 1248, 1157, 1039, 985, 897, 832. LC-MS (ESI) m/z calculated for C₁₂H₈F₃NO₃ [M+H]⁺ 272.05, found [M+H]⁺ 272.06.

N-(6-methoxypyridin-3-yl) octanamide (13): white solid (96 mg, 48% yield). Melting point: 61.2-61.7° C. UVₘₐₓ (nm):278. ¹H NMR (CDCl₃): 8.12 (d, J=2.70, 1H, pyridine), 7.91 (dd, J₁=2.70, J₂=9.00, 1H pyridine), 7.10 (s, 1H, CONH), 6.70 (d, J=9.00, 1H pyridine), 3.90 (s, 3H, OCH₃), 2.35 (t, J=6.90, 2H, CH₂CO), 1.72 (q, J=6.90, 2H, CH₂), 1.20-1.40 (m, 8H, 4-CH₂), 0.87 (t, J=6.30, 3H, CH₃). ¹C NMR (CDCl₃): 172, 161, 139, 133, 129, 111, 54, 37, 32, 29, 29, 26, 22, 20, 14; GC/MS EI m/z: 250 (M⁺, 16%), 124(100%), 95 (16%). IR (KBr) νₘₐₓ (cm⁻¹): 3291 (s, NH), 3051, 2932, 2858, 1657 (s, C═O), 1581, 1535, 1496, 1372, 1284, 1189, 1026, 968, 913, 830. LC-MS (ESI) m/z calculated for C₁₄H₂₂N₂O₂ [M++]⁺ 251.17, found [M+H]⁺ 251.15.

N-(6-methoxy-2-methylpyridin-3-yl) octanamide (14): white solid (130 mg, 86% yield). Melting point: 67.4-68.2° C. UVₘₐₓ (nm): 276. ¹H NMR (CDCl₃): 7.77 (d, J=8.70, 1H, pyridine), 6.81 (s, 1H, CONH), 6.57 (d, J=8.70, 1H pyridine), 3.89 (s, 3H, OCH₃), 2.37 (s, 3H, CH₃), 2.35 (t, J=6.90, 2H, CH₂CO), 1.72 (p, J=6.90, 2H, CH₂), 1.20-1.40 (m, 8H, 4-CH₂), 0.88 (t, J=6.30, 3H, CH₃). ¹C NMR (CDCl₃): 172, 161, 149, 136, 125, 108, 54, 37, 32, 29, 29, 26, 22, 20, 14; GC/MS EI m/z: 264 (M⁺, 33%), 180 (33%), 138 (100%), 57 (24%). IR (KBr) νₘₐₓ (cm⁻¹): 3272 (s, NH), 2928, 2859, 1647 (s, C═O), 1595, 1527, 1474, 1422, 1318, 1259, 1193, 1109, 1041, 993, 825. LC-MS (ESI) m/z calculated for C₁₅H₂₄N₂O₂ [M+H]⁺ 265.19, found [M+H]⁺ 265.16.

N-(4-(trifluoromethyl)-2-oxo-2H-chromen-7-yl) octanamide (15): white solid. Melting point: 136.6-137.1° C. UVₘₐₓ (nm):392. ¹H NMR (CDCl₃): 7.80 (d, J=2.10, 1H, Ar), 7.53-7.68 (m, 2H, Ar), 6.69 (s, 1H, CH), 2.43 (t, J=7.50, 2H, CH₂), 1.75 (m, 2H, CH₂), 1.22-1.42 (m, 8H, 4-CH₂), 0.88 (t, J=7.20, 3H, CH₃). ¹C NMR (CDCl₃): 172, 166, 160, 160, 155, 143, 126, 117, 114, 109, 107, 38, 32, 29, 29, 26, 22, 14; GC/MS EI m/z: 355 (M⁺, 7%), 271 (9%), 229 (100%), 201 (35%), 127 (6%), 57 (40%). IR (KBr) νₘₐₓ (cm⁻): 3310 (s, NH), 3107, 2925, 2861, 1718(s, C═O), 1619, 1585, 1524, 1409, 1358, 1278, 1158, 1008, 954, 867, 822. LC-MS (ESI) m/z calculated for C₁₈H₂₀F₃NO₃ [M+H]⁺ 356.14, found [M+H]⁺356.12.

N-(4-nitrophenyl) octanamide (16): white solid. Melting point: 80.9-81.6° C. UVₘₐₓ (nm):320. NMR (CDCl₃): 8.20 (dd, J₁=9.00, J₂=1.50, 2H, Ar), 7.70 (dd, J₁=9.00, J₂=1.50, 2H, Ar), 7.50 (brs, 1H, NH), 2.40 (t, J=8.10, 2H, CH₂), 1.70 (quint, J=6.90, 2H, CH₂), 1.23-1.40 (m, 8H, 4 CH₂), 0.87 (t, J=7.20, 3H, CH₃). ¹C NMR (CDCl₃): 172, 144, 143, 125, 125, 119, 119, 38, 32, 29, 29, 25, 22, 14; GC/MS EI m/z: 264 (M⁺, 4%), 180 (28%), 138 (100%), 127 (26%), 57 (70%). IR (KBr) νₘₐₓ (cm⁻¹): 3315 (s, NH), 31160, 3095, 2930, 2859, 1683 (s, C═O), 1606, 1553 (s, NO₂), 1505, 1466, 1404, 1333 (s, NO₂), 1255, 1171, 1110, 1027, 956, 855. LC-MS (ESI) m/z calculated for C₁₄H₂₀N₂O₃ [M+H]⁺ 265.15, found [M+H]⁺ 265.14.

Preparation of substrate (17). Arachidonic acid (200 mg, 0.65 mmol) was added to an ice-cooled CH₂Cl₂ solution (10 ml) containing triethylamine (0.65 mmol) and 1-[3-(dimethylamino)propyl]-3-ethylcarbodiimide hydrochloride (139 mg, 0.73 mmol) under nitrogen protection. After a 20 min reaction under an ice-cooled bath, a dichloromethane solution (2 ml) containing 5-amino-2-methoxypyridine (65 mg, 0.65 mmol) was added to the reaction. The reaction was stirred for 3 hours under an ice-cooled bath. The reaction mixture was washed twice with 1 M sodium bicarbonate solution (30 ml) and saturated NaCl solution (30 ml). The combined organic phase was dried under magnesium sulfate for 1 hr. After filtration and evaporation, it gave an oily product, which was chromatographed with a gradient of hexane and ethyl acetate. A slight yellowish oil (46 mg) was produced (17% yield). For all procedures described here, aluminum foil was used to protect the solution from being exposed to light. UVₘₐₓ (nm):246. ¹H NMR (CDCl₃): 8.12 (d, J=2.70, 1H, pyridine), 7.94 (dd, J₁=2.70, J₂=9.00, 1H pyridine), 7.09 (s, 1H, CONH), 6.72 (d, J=9.00, 1H pyridine), 5.30-5.46 (m, 8H, 4H—C═C—H), 3.90 (s, 3H, OCH₃), 2.80-2.84 (s, 6H, 3CH₂), 2.36 (t, J=7.50, 2H, CH₂), 2.17 (quint, J=7.20, 2H, CH₂), 2.07 (q, J=7.20, 2H, CH₂), 1.83 (q, J=7.20, 2H, CH₂), 1.73 (quint, J=7.20, 2H, CH₂), 1.27-1.40 (6H, 3CH₂), 0.88 (t, J=6.90, 3H, CH₃). ¹C NMR (CDCl₃): 172, 161, 139, 133, 131 (130.8:130.7=2:1), 129.3 (129.29:129.27=2:1), 129.2, 129.0, 128.9 (128.9:128.8=2:1), 128.5, 128.4, 128.3 (128.25:128.34=2:3), 128.1 (128.10:128.06=2:3), 127.8 (127.77:127.73=1:2), 37, 32, 30, 27.5, 26.8, 26.7, 25.9, 25.5, 24.70, 23, 14; GC/MS EI m/z: 410 (58%), 166 (84%), 124(100%), 79 (54%), 55 (42%). IR (film) νₘₐₓ (cm⁻¹): 3320 (w, NH), 3011, 2927, 2870, 1659 (m, C═O), 1570, 1535, 1494, 1450, 1383, 1273, 1029, 911, 830. LC-MS (ESI) m/z calculated for C₂₆H₃₈N₂O₂ [M+H]⁺ 411.30, found [M+H]⁺ 411.26.

Preparation of recombinant human FAAH. Transgenic expression of the human FAAH in a baculovirus system was performed by following the method described in Nishi et al., Arch. Biochem. Biophys., 445:115-123 (2006). The cDNA encoding human FAAH (GenBank Accession No. NM_{—}001441) was amplified by PCR using a human liver cDNA library (Invitrogen; Carlsbad, Calif.) as a template. The primer pair was 5′-AGATCTATGGTGCAGTACGAGCTGTGGGCC-3′ and 5′-GAATTCTCAGGATGACTGCTTTTCAGGGGT-3′. BglII and EcoRI endonuclease sites (underlined) were incorporated just upstream of the start codon and downstream of the stop codon of the coding sequence of FAAH, respectively. The PCR products were cloned into a pCR2.1 vector (Invitrogen) and the nucleotide sequence was verified by DNA sequencing. The cDNA fragments were then excised and directionally ligated to the BglII and EcoRI sites of the baculovirus transfer vector pAcUW21 (Lopez-Ferber et al., Baculovirus transfer vectors, in “Baculovirus Expression Protocols,” Richardson (Ed.), pp 25-63, Humana Press, Totowa, N.J., 1995). Recombinant baculoviruses harboring the human FAAH gene, Ac-hFAAH, were generated by co-transfection of Spodoptera frugiperda-derived Sf21 cells with the recombinant transfer vector plasmid and Bsu36I-cleaved BacPAK6 viral DNA (Clontech Laboratories; Mountain View, Calif.) as described in O'Reilly et al., Baculovirus Expression Vectors: A Laboratory Manual, Oxford University Press, New York, 1992. Trichoplusia ni-derived High Five cells (1×10⁶ cells/ml) were inoculated with a high titer of Ac-hFAAH. At 72 hours post-infection, the infected cells were harvested by centrifugation at 2,000×g for 20 min at 4° C. and suspended in 50 mM Tris-HCl (pH 8.0) containing 150 mM NaCl, 1 mM EDTA, 1 μM pepstatin, 100 μM leupeptin, and 0.1 mg/ml aprotinin. The cell suspension was then homogenized using a Polytron homogenizer and centrifuged at 10,000×g for 20 min at 4° C. The microsomal fraction was collected by ultracentrifugation of the supernatant at 100,000×g for 60 min at 4° C. The pellet was resuspended in 20 mM Tris-HCl (pH 8.0) containing 10% (w/v) glycerol and 1% (w/v) Triton-X 100 and stored at −80° C. until use.

Molar extinction coefficient. According to Beer's law, A_{λ}=ε*C*L, where A_{λ} is the absorbance at wavelength (λ nm); ε is called the molar extinction coefficient; L is the width of the sample cuvette; and C is the concentration of the solute. In this example, a series of concentrations falling between 10 and 50 μM (final concentration) for each fluorescent reporter was used for determination of the molar extinction coefficient. Assays were performed in a quartz cuvette (1 cm optical passage). 10 μl of a stock solution of a reporter in ethanol (1, 2, 3, 4, or 5 mM) was added to 990 μl H₂O. The resulting mixture was degassed by sonication with ultrasonic cleaner Model 750 (VWR International; Wester, Pa.) for 10 seconds at a power level of 9. Measurements were performed with a Spectra Max M2 spectrophotometer (Molecular Devices; Sunnyvale, Calif.) at a wavelength of 290 nm. A slope (or extinction coefficient) was obtained by using an average absorbance of triplicate samples.

Maximal excitation and emission wavelengths. A stock solution in ethanol of each compound (10 μl, 5 mM) was added to 990 μl of sodium phosphate buffer (0.1 M, pH 8.0, final substrate concentration 50 μM) in a 1-cm quartz cuvette. The quartz cuvette was scanned from 200 to 500 nm for absorption spectra with a Spectra Max M2 spectrophotometer. Maximal excitation and emission wavelengths were obtained from scanning from 250 to 600 nm and 360 to 600 nm by a 2 nm interval at 30° C., respectively.

Quantum yield. The fluorescence quantum yield, φ_{f}, was calculated according to the following equation:

Ø_{F}=Ø_{std}*(I/I_{std})*(OD_{std}/OD)*(n/n_{std})²,

wherein Ø_{F} is the quantum yield of the tested fluorescent reporter; Ø_{std} is the fluorescence quantum yield of the standard; I and I_{std} are the integrated emission intensities of the sample and the standard, respectively; OD and OD_{std} are the absorbance of the sample and standard, respectively, at the desired wavelength λₑₓ; and n and n_{std} are the indexes of refraction of the sample and standard solutions, respectively (Horspool and Song (Eds.), CRC Handbook of Organic Photochemistry I, pp 234-235, CRC Press, Boca Raton, Fla., 1995).

In this example, 3-aminopyridine in water at the maximal excitation wavelength of 290 nm was used as a standard (Ø_{std}=0.32, εₘₐₓ=2800 M⁻¹ cm⁻¹) (see, e.g., Weisstuch et al., J. Phys. Chem., 72:1982-1987 (1968)). In order to minimize inner filter effects, the optical densities of samples at the excitation wavelength (λ=290 nm) were kept in the range of 0.095-0.13 (less than 0.150-0.165). The final concentration of each tested sample in a quartz cuvette was 10 μM with 1% ethanol in water, same as that for the standard. The integrated area of fluorescent intensity for the standard or the samples was based on the area between 360 and 500 nm. The difference of the index of refraction between the standard and the sample was neglected because (1) all measurements were performed in the same quartz cuvette and spectrofluorimeter; (2) the final concentrations of the sample and the standard were the same; and (3) the structures of the sample and standard were very similar, i.e., only one pyridine ring. The quantum yields presented in Table 1 were based on calculation of the average of OD and integrated fluorescent intensities of triplicates. The standard deviation for each quantum yield was less than ±5%.

The dependence of relative fluorescent intensity on pH values. 5-amino-2-methoxypyridine (6) was used to determine whether protonization/de-protonization affects the relative fluorescent intensities of the reporters. A stock solution of (6) in ethanol (5 μl, 5 mM) was added to 995 μl 0.1 M sodium phosphate buffer (from pH 6.0 to pH 8.0) or 0.1 M Tris/HCl buffer (pH 9.0) in a quartz cuvette (final substrate concentration 25 μM). Relative fluorescent unit/intensity (RFU) for emission spectra was recorded by a 2 nm interval at room temperature using a fixed excitation wavelength (λ=302 nm). The RFU of each pH value was an average of triplicates and the standard deviation for each point was less than 1%. Similarly, the RFU of the excitation spectrum was recorded by a 2 nm interval at room temperature using a fixed emission wavelength (λ=396 nm) in 0.1 M phosphate buffer (pH 8.0).

The effects of protein amounts on the relative fluorescent intensity. 5-amino-2-methoxypyridine (6) was used to examine the effect of protein levels on the relative fluorescent intensities of the aminopyridines. In brief, different concentrations of (6) (final concentration: 0, 0.4, 0.78, 1.56, 3.13, 6.25, 12.5, or 25 μM) were separately mixed with different levels of bovine serum albumin (BSA; 0, 1, 10, or 100 μg/well) in 200 μl of 0.1 M sodium phosphate buffer (pH 8.0). Relative fluorescent intensity at 37° C. was recorded at an excitation wavelength (302 nm) and emission wavelength (396 nm). Similar methods were used to determine the effects of relative fluorescent intensities of 5-amino-2-methoxy-6-methylpyridine (8) and 7-amino-4-trifluoromethylcoumarin (9) on the protein levels.

Comparison of aqueous solubility of substrates. The aqueous solubility of the aminopyridine derivatives (e.g., 13, 14) and the 7-aminocoumarin derivative (e.g., 15) was determined in clear 96-well styrene flat-bottom microtiter plates with a Spectra Max M2 spectrophotometer. Different concentrations of the substrates (13, 14 and 15; final concentration: 0.39, 0.78, 1.56, 3.13, 6.25, 12.5, 25, 50, 100, or 200 μM) in 200 μl 0.1 M sodium phosphate buffer (1% ethanol; pH 8.0) were used and the absorbance was recorded at a wavelength of 800 nm at 37° C. The measurements were performed in three replicates for each concentration.

Comparison of the lowest detection limits of protein concentration of FAAH toward colorimetric and fluorescent substrates. In general, all assays were performed in 96-well styrene flat-bottom microtiter plates with a Spectra Max M2 spectrophotometer at 37° C. for 10 mM. The total volume of each well was 201 μl consisting of 0.1 M sodium phosphate buffer (pH 8.0) (190 μl) containing 1% glycerol and 0.1% Triton-X 100, stock or diluted protein solution (10 μl), and the substrate solution (1 μl) in ethanol (final concentration 200 μM for 10, 11, 13 or 14) or in DMSO (final concentration 25 μM for 12, 15, or 16; 50 μM for 17). The lowest detection limits of microsomal fatty acid amide hydrolase (FAAH) by substrates were determined by the fact that the signal at 10 min after kinetic measurements are equal to or over three times the noise or basal line at 0 min. Colorimetric substrate (16) assays were performed in clear 96-well microtititer plates using an absorbance wavelength at 382 nm. The fluorescent substrates containing 5-amino-2-methoxypyridine (e.g., 10, 13) were measured at an excitation wavelength (302 nm), emission wavelength (396 nm), and auto cutoff wavelength (325 nm). Similarly, the fluorescent substrates containing 5-amino-2-methoxy-6-methylpyridine (e.g., 11, 14, and 17) were measured at an excitation wavelength (304 nm), emission wavelength (392 nm), and an auto cutoff wavelength of 325 nm. In addition, an excitation wavelength (366 nm), emission wavelength (496 nm), and auto cutoff wavelength (495 nm) were used for the substrates (e.g., 12 and 15) containing 7-amino-4-trifluoromethyl coumarin.

Comparison of hydrolytic ratios of different substrates by FAAH with time course. To further compare the hydrolytic ratios of different substrates by FAAH, microsomal FAAH was incubated with different substrates at 37° C. for 90 min and hydrolysis was recorded at 2-min intervals. Assays were performed by the following procedures: a substrate solution (1 μl, 10 mM in DMSO) was added to protein-containing buffer (200 μl). This buffer contained 0.1 M phosphate buffer (pH 7.4) (or 0.1 M Tris/HCl buffer (pH 9.0)), 1% glycerol, 0.1% Tritron X-100, 2.5% DMSO, and FAAH microsomes (0.67 μg for substrates 13, 14, and 17; 100 μg for substrates 15 and 16). The final concentration of all examined substrates was 50 μM. The excitation and emission wavelengths of measurements are described above.

Specific activity determination. The assays for specific activities were performed as described above for the sensitivity assay. The specific activity presented is based on the following: 1) the signal after a 10 min enzymatic reaction was at least 3 fold the baseline signal at 0 min, and 2) the amount of hydrolyzed substrate during a 10 min period was less than 10% of the total amount of the substrate added. To correct for protein-induced fluorescence quenching, a standard curve of 5-amino-2-methoxypyridine (6) or 5-amino-2-methoxy-6-methylpyridine (8) was constructed in the presence of the same protein concentration used for the assay of that enzyme.

Kinetic study. The kinetic assays were also performed as described above for the sensitivity assay. Briefly, a total volume of 201 μl contained 0.1 M sodium phosphate buffer (pH 8.0) (190 μl) with 1% glycerol and 0.1% Triton-X 100, protein solution (10 μl), and substrate solution (1 μl). The protein content was 0.67 μg per well (201 μl). The concentration of substrate (13) in DMSO was 200, 100, 50, 25, 12.5, 6.25, 3.13, 1.56, or 0.78 μM. Similarly, the concentration of substrate (17) in DMSO was 100, 50, 25, 12.5, 6.25, 3.13, 1.56, 0.78, or 0.39 μM. Four replicates were applied for each concentration. Reported kinetic data (kₘ and Vₘₐₓ) were analyzed by the SigmaPlot Enzyme Kinetics software (2001) using the Michaelis-Menten equation for all concentrations.

Statistical treatment of the results. Student's T-test (p≦0.05) was used to evaluate whether a difference between two means is significant at the given probability level in Table 4, where a comparison between control microsomes from non-infected High Five cells and microsomes from cells infected by FAAH-expressing virus or between human liver S9 and human liver microsomes was determined. The other data presented were calculated using Microsoft Excel for average and standard deviation.

### Example 2

### Highly Sensitive Fluorescent Assays for Aminopeptidases

This example describes the development of novel and highly sensitive fluorescent substrates for aminopeptidases that are based on substituted aminopyridines. Aminopeptidases are a class of enzymes that hydrolyze the N-terminal peptidase bond in proteins and peptides. They have a broad substrate specificity and are widely distributed in many tissues and cells in animals, bacteria, viruses, and plants. L-leucine aminopeptidase is one of the best studied aminopeptidases. It is of significant biological and medical importance because its altered activity is observed in multiple diseases such as cancer, eye lens aging, and cataracts. It may also play an important role in the early events of HIV infection and thus serum L-leucine aminopeptidase activity may be a useful marker of HIV infection and response to chemotherapy (Grembecka et al., Mini Rev. Med. Chem., 1:133-144 (2001)). Highly sensitive assays are necessary to identify inhibitors of aminopeptidases. Currently, the majority of assays for inhibitor screening is based on the colorimetric detection of substrates containing para-nitroaniline (see, e.g., Stockel-Maschek et al., Bioorg. Med. Chem., 13:4806-4820 (2005); Grembecka et al., Med. Chem., 46:2641-2655 (2003)). However, these assays lack the appropriate sensitivity for use in high-throughput screening approaches. Thus, the fluorescent aminopeptidase substrates described herein provide a valuable tool for use in assays of aminopeptidase activity and to screen for aminopeptidase inhibitors by high-throughput assays.

### Results

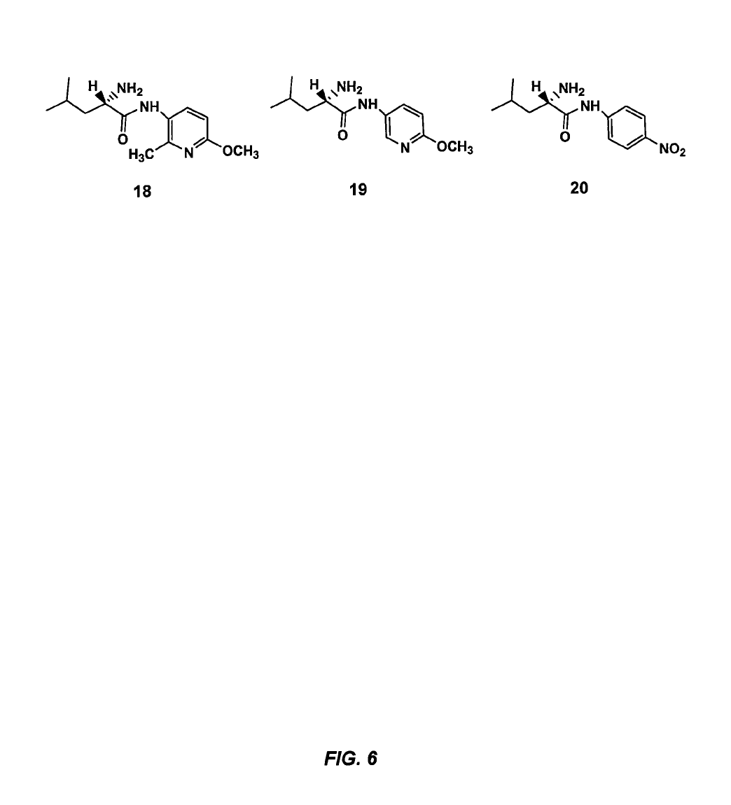

The structures of the fluorescent and colorimetric aminopeptidase substrates used in this example are provided in FIG. 6. As shown in Table 5, the results indicated that substrate (19) was at least 100 times more sensitive than substrate (20), which is currently extensively used for screening for inhibitors of L-Leucine aminopeptidase. The dramatic difference between substrates (18) and (19) also indicated that L-Leucine aminopeptidase is very strict with the structures of reporters.

TABLE 5
Comparison of the sensitivity of L-Leucine aminopeptidase
from porcine kidney microsomes toward the colorimetric
substrate (20) and fluorescent substrates (18) and (19).

| | Protein | | Signal | Average | |
|---|---|---|---|---|---|
| Substrate | (μg/200 μL) | | (OD or RFU/sec) | (OD or RFU/sec) | St Dev |
| (18) | 1.97 | base line | 169 163 160 165 | 164 | 4.0 |
| | | signal | 491 513 501 471 | 494* | 17.6 |
| (19) | 0.018 | base line | 64 62 74 70 67 | 5.6 | |
| | | signal | 221 249 199 242 | 228* | 22.3 |
| (20) | 1.97 | base line | 0.07 0.09 0.08 0.08 | 0.08 | 0.01 |
| | | signal | 0.25 0.24 0.23 0.25 | 0.24* | 0.01 |

Assays were performed in 190 μl of 0.1 M sodium phosphate buffer (pH 7.2), 10 μl diluted protein solution, and 1 μl of 5 mM substrate in ethanol (final conc. 25 μM) at 37° C. Absorbance wavelength for (20) was 382 nm. Excitation and emission wavelengths for (18) and (19) were 302 and 396, and 304 and 392, respectively.

### Materials and Methods

Chemicals and enzymes. Fluorescent reporters including 5-amino-2-methoxypyridine and 3-amino-6-methoxy-2-picoline were purchased from Asychem (Durham, N.C.). L-Leucine-p-nitroanilide (20) and microsomal L-Leucine aminopeptidase (L5006) were purchased from Sigma-Aldrich (Saint Louis, Mo.).

Structural identification was based on data from proton nuclear magnetic resonance (¹H-NMR) and gas chromatography/mass spectrometry (GC/MS). Proton NMR spectra were acquired from a Mercury 300 spectrometer (Medical Systems, Inc.; Palo Alto, Calif.). Chemical shift values are given in parts per million (ppm) downfield from the internal standard (trimethylsilane). Chemical purity of the final products was supported by the spectra described above, a single spot on TLC at a wavelength of 254 nm, and lack of fluorescence from 5-amino-2-methoxypyridine or 5-amino-2-methoxy-6-methylpyridine on TLC at a wavelength of 254 nm. Melting points were determined on an OptiMelt Automated Melting Point System (Stanford Research Systems; Sunnyvale, Calif.).

Preparation of L-leucine aminopeptidase substrates. To an ice-cooled solution containing N-(tert-butoxycarbonyl)-L-Leucine monohydrate (0.49 g), anhydrous CH₂Cl₂ (10 ml), and triethylamine (0.2 g), 1-[3-(dimethylamino)propyl]-3-ethylcarbodiimide hydrochloride (0.393 g) and 5-amino-2-methoxypyridine (0.24 g) were added. The reaction was warmed to room temperature overnight. The solution was diluted with ethyl acetate (50 ml) and then washed sequentially with a saturated sodium bicarbonate solution and a sodium chloride solution. The organic phase was dried by magnesium sulfate. A crude solid product was yielded after it was filtrated and evaporated under reduced pressure. Chromatography was performed on the crude product with hexane and ethyl acetate (3:1), which yielded the pure protected substrate (18A) (0.5 g). Substrate (18A) was then de-protected by dry HCl. Briefly, acetyl chloride (5 eqv) was added into the solution containing the pure protected substrate (0.5 g), ethanol (10 eqv), and ethyl acetate (20 ml). The reaction lasted 30 min at room temperature. The resulting mixture was washed by saturated sodium bicarbonate and extracted with ethyl acetate (3×30 ml). The combined organic phase was dried by magnesium sulfate. Crude red oil was yielded after it was filtrated and evaporated under reduced pressure. The crude oil was chromatographed with methanol and ethyl acetate (1:9) and yielded a slightly red oil (18) (0.16 g). A similar procedure was used to prepare the compounds (19A) and (19).

tert-Butyl (S)-1-(6-methoxy-2-methylpyridin-3-ylcarbamoyl)-3-methylbutyl carbamate (18A): white solid (0.49 g, 94% yield). Melting point: 148.0-148.7° C. ¹H NMR (CDCl₃): 7.93 (br, 1H, CONH), 7.87 ((d, J=9.0, 1H, pyridine), 6.56 (d, J=9.0, 1H, pyridine), 4.89 (d, J=7.80, 1H, CH), 3.89 (s, 3H, OCH₃), 2.38 (s, 3H, CH₃), 1.66-1.4 (3H, CH₂CH), 1.47 (s, 9H, 3CH₃), 0.98 (t, J=6.00, 6H, 2CH₃).

N-(6-methoxy-2-methylpyridin-3-yl), (S)-2-amino-4-methylpentanamide (18): slightly red oil. ¹H NMR (CDCl₃): 9.38 (s, 1H, CONH), 8.12 (d, J=9.00, 1H, pyridine), 6.58 (d, J=9.00, 1H pyridine), 3.89 (s, 3H, OCH₃), 3.56 (t, J=10.5, 1H, CHNH₂), 2.41 (s, 3H, CH₃), 1.75-1.86 (3H, CH₂CH), 0.99 (d, J=6.30, 3H, CH₃), 0.97 (d, J=6.30, 3H, CH₃).

tert-Butyl (S)-1-(6-methoxypyridin-3-ylcarbamoyl)-3-methylbutylcarbamate (19A): white solid (0.5 g, 74% yield). Melting point: 160.1-163.5° C. ¹H NMR (CDCl₃): 8.40 (s, br, 1H, CONH), 8.17 (d, J=2.70, 1H, pyridine), 7.85 (dd, J₁=2.70, J₂=8.70, 1H pyridine), 6.70 (d, J=8.70, 1H pyridine), 4.96 (d, J=7.80, 1H, CH), 3.90 (s, 3H, OCH₃), 1.45 (s, 9H, 3CH₃), 1.66-1.79 (3H, CH₂CH), 0.96 (t, J=6.00, 6H, 2CH₃).

N-(6-methoxypyridin-3-yl), (S)-2-amino-4-methylpentanamide (19): slightly red oil. ¹H NMR (CDCl₃): 9.47 (s, 1H, CONH), 8.23 (d, J=2.40, 1H, pyridine), 8.01 (dd, J₁=2.70, J₂=8.70, 1H pyridine), 6.72 (d, J=8.70, 1H pyridine), 3.91 (s, 3H, OCH₃), 3.52 (t, J=10.5, 1H, CHNH₂), 1.75-1.86 (3H, CH₂CH), 0.99 (d, J=6.30, 3H, CH₃), 0.97 (d, J=6.30, 3H. CH₃).

Comparison of the sensitivities of L-Leucine aminopeptidase toward colorimetric and fluorescent substrates. In general, all assays were performed in 96-well styrene flat-bottom microtiter plates using a Spectra Max M2 spectrophotometer at 37° C. for 10 min. The total volume of each well was 201 μl containing 0.1 M sodium phosphate buffer (190 μl, pH 7.2), stock or diluted protein solution (10 μl), and 5 mM of the substrate solution (1 μl) in either ethanol for substrates (18) and (19). The lowest detection limit of microsomal L-Leucine aminopeptidase by substrates was determined by the fact that the signal at 10 min was equal to or over 3 times the basal line at 0 min. Colorimetric substrates assays (e.g., (20)) were performed in clear 96-well microtititer plates using an absorbance wavelength of 382 nm. The fluorescent substrates containing 5-amino-2-methoxypyridine (e.g., (19)) were measured at an excitation wavelength (302 nm), emission wavelength (396 nm), and auto cutoff wavelength (325 nm). Similarly, the fluorescent substrates containing 5-amino-2-methoxy-6-methylpyridine (e.g., (18)) were measured at an excitation wavelength (304 nm), emission wavelength (392 nm), and auto cutoff wavelength (325 nm).

### Example 3

### Red-Shifted Substituted Aminopyridines

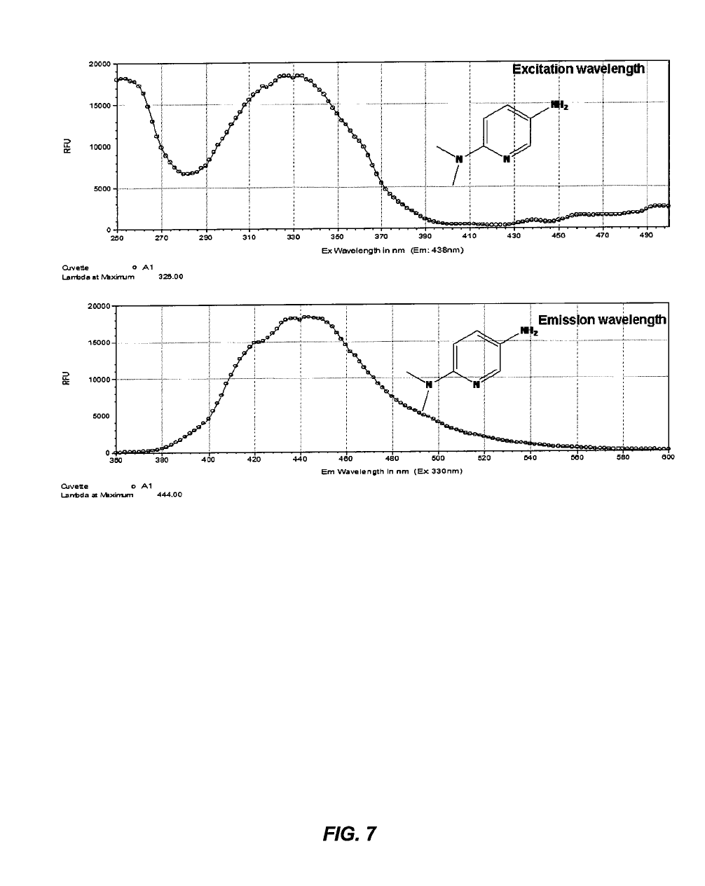

This example describes the development of substituted aminopyridines with optical properties that are shifted towards the red end of the electromagnetic spectrum. As shown in FIG. 7, the presence of an electron-donor group such as a dimethylamino group para to the amine group on the aminopyridine produces a substituted aminopyridine with a red-shifted spectrum. For example, substitution of a methoxy group for a dimethylamino group shifts the excitation wavelength from 302 nm to 330 nm and the emission wavelength from 396 nm to 444 nm.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A conjugate comprising:
(a) a substituted aminopyridine having the formula: 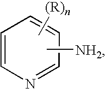
wherein R is n independently selected substituted groups and n is 1, 2, 3, or 4; and
(b) an organic molecule covalently attached to the —NH₂ group of the substituted aminopyridine via an amide bond.

2. The conjugate of claim 1, wherein at least one substituted group comprises an alkoxy group.

3. The conjugate of claim 2, wherein the alkoxy group is a methoxy group.

4. The conjugate of claim 3, wherein the methoxy group is para to the —NH₂ group.

5. The conjugate of claim 1, wherein n is 1.

6. The conjugate of claim 1, wherein the conjugate is a substrate for an amide hydrolase.

7. The conjugate of claim 6, wherein the organic molecule comprises a fatty acid.

8. The conjugate of claim 7, wherein the amide hydrolase comprises a fatty acid amide hydrolase (FAAH).

9. The conjugate of claim 6, wherein the organic molecule comprises an amino acid, a peptide, a polypeptide, or a protein.

10. The conjugate of claim 9, wherein the amide hydrolase comprises a peptide hydrolase.

11. The conjugate of claim 10, wherein the peptide hydrolase comprises an aminopeptidase.

12. A method for determining amide hydrolase activity, the method comprising:
(a) contacting an amide hydrolase with the conjugate of claim 1; and
(b) measuring a level of substituted aminopyridine released from the conjugate by the amide hydrolase.

13. The method of claim 12, wherein the level of released substituted aminopyridine is measured using fluorescence detection.

14. The method of claim 12, wherein the level of released substituted aminopyridine is associated with a disease or disorder.

15. The method of claim 14, wherein the disease or disorder is selected from the group consisting of a neurological disorder, an inflammatory disease, an autoimmune disease, a circulatory disease, a liver disease, and cancer.

16. A method for identifying a compound that inhibits an amide hydrolase, the method comprising:
(a) contacting an amide hydrolase with the conjugate of claim 1 and a compound; and
(b) determining the effect of the compound on amide hydrolase activity, thereby identifying a compound that inhibits the amide hydrolase.

17. The method of claim 16, wherein the effect of the compound on amide hydrolase activity is determined by measuring a level of substituted aminopyridine released from the conjugate by the amide hydrolase.

18. The method of claim 17, wherein the level of released substituted aminopyridine is measured using fluorescence detection.

19. The method of claim 17, further comprising comparing the level of released substituted aminopyridine in the presence of the compound relative to the absence of the compound.

20. The method of claim 19, wherein a decrease in the level of released substituted aminopyridine indicates that the compound inhibits the amide hydrolase.

21. A method of inhibiting an amide hydrolase in a subject, the method comprising:
administering to the subject a therapeutically effective amount of a compound identified by the method of claim 16.

22. A kit comprising the conjugate of claim 1 and directions for use of the conjugate in determining amide hydrolase activity.

23. A kit comprising the conjugate of claim 1 and directions for use of the conjugate in identifying a compound that inhibits an amide hydrolase.
